(19) United States
(12) Patent Application Publication
(10) Pub. No.: US 2025/0067827 A1
(43) Pub. Date: Feb. 27, 2025

(54) SYSTEMS AND METHODS FOR INTERFERENCE SIGNAL COLLECTION AND PROCESSING

(71) Applicant: SHANGHAI UNITED IMAGING HEALTHCARE CO., LTD., Shanghai (CN)

(72) Inventors: Qian LIU, Shanghai (CN); Bo LI, Shanghai (CN); Rongxing ZHANG, Shanghai (CN); Yuanding BAO, Shanghai (CN); Aolin LIU, Shanghai (CN); Jianxing WU, Shanghai (CN); Fuyi FANG, Shanghai (CN)

(73) Assignee: SHANGHAI UNITED IMAGING HEALTHCARE CO., LTD., Shanghai (CN)

(21) Appl. No.: 18/940,801

(22) Filed: Nov. 7, 2024

Related U.S. Application Data
(63) Continuation of application No. PCT/CN2023/096984, filed on May 30, 2023.

(30) Foreign Application Priority Data
May 30, 2022 (CN) 202210599763.7
Aug. 9, 2022 (CN) 202222088624.6
Aug. 9, 2022 (CN) 202222093262.X

(51) Int. Cl. G01R 33/56; G01R 33/565; G06T 11/00

(52) U.S. Cl. CPC G01R 33/5608; G01R 33/5659; G06T 11/006; G06T 2211/441

(57) ABSTRACT

Systems and methods for interference signal collection and processing are provided. The systems may obtain initial signals and first interference signals collected in at least one first time window during an MR scan of a target subject. The nuclei in the target subject may be in an excited state in the at least one first time window. The initial signals may be collected by a receiving coil of an MRI device, and the first interference signals may be collected by an interference signal acquisition device. The systems may also determine second interference signals collected by the receiving coil in the at least one first time window based on the first interference signals. The systems may further determine imaging signals collected by the receiving coil in the at least one first time window by correcting the initial signals based on the second interference signals.

### CROSS-REFERENCE TO RELATED APPLICATIONS

This application is a continuation of International Patent Application PCT/CN2023/096984, filed on May 30, 2023, which claims priority of Chinese Patent Application No. 202210599763.7, filed on May 30, 2022, Chinese Patent Application No. 202222088624.6, filed on Aug. 9, 2022, and Chinese Patent Application No. 202222093262.X, filed on Aug. 9, 2022, the contents of each of which are hereby incorporated by reference.

### TECHNICAL FIELD

The disclosure generally relates to magnetic resonance imaging technology, and more particularly relates to systems and methods for interference signal collection and processing in magnetic resonance imaging (MRI).

### BACKGROUND

Magnetic resonance imaging (MRI) devices can obtain images of arbitrary sections of a human body non-invasively. During imaging processes, an MRI device receives MR signals excited by a radio frequency (RF) signal for generating MR images. External electromagnetic interference sources caused by various devices and/or human factors in the unshielded environment would affect the MRI signals, thereby reducing the image quality of the MR images. Thus, the MRI device usually needs to work under a shielding cage (or a shielding room). The manufacture of the shielding cage is costly (e.g., usually accounting for more than 10% of the cost of an MRI system) and the shielding cage occupies a large space. Though the fringing field for an MRI device with ultra-low field (the main magnetic field strength <0.1 teslas) is reduced, which eliminates the need for a magnetic shielding cage, a radio frequency (RF) shielding cage is still needed to eliminate external electromagnetic interference signals during scanning. However, the RF shielding cage used in the MRI device is often unable to realize the shielding of interference signals at the head and tail of detection channels, and thus cannot eliminate the interference of the interference signals on the quality of MR images. Therefore, it is desirable to provide systems and methods for interference signal collection and/or processing, thereby reducing the interference of interference signals on the MR images for improving the image quality of the MR images.

### SUMMARY

In one aspect of the present disclosure, a method, implemented by a computing device including a storage device and at least one processor, is provided. The method may include obtaining initial signals and first interference signals collected in at least one first time window during a Magnetic Resonance (MR) scan of a target subject. The nuclei in the target subject may be in an excited state in the at least one first time window. The initial signals may be collected by a receiving coil of a Magnetic Resonance Imaging (MRI) device. The first interference signals may be collected by an interference signal acquisition device. The method may also include determining second interference signals collected by the receiving coil in the at least one first time window based on the first interference signals. The method may further include determining imaging signals collected by the receiving coil in the at least one first time window by correcting the initial signals based on the second interference signals.

In another aspect of the present disclosure, a coil assembly for Magnetic Resonance Imaging (MRI) is provided. The coil assembly may include a housing forming an accommodation space configured to accommodate a target subject. The coil assembly may also include a receiving coil configured to collect initial signals during an MR scan of the target subject. The coil assembly may further include an interference signal acquisition device configured to collect interference signals during the MR scan. The interference signals may be used to correct the initial signals to determine imaging signals collected during the MR scan.

In some embodiments, a system is provided. The system may include at least one storage device including a set of instructions, and at least one processor in communication with the at least one storage device. When executing the set of instructions, the at least one processor may cause the system to perform following operations. The system may obtain initial signals and first interference signals collected in at least one first time window during a Magnetic Resonance (MR) scan of a target subject. The nuclei in the target subject may be in an excited state in the at least one first time window. The initial signals may be collected by a receiving coil of a Magnetic Resonance Imaging (MRI) device. The first interference signals may be collected by an interference signal acquisition device. The system may also determine second interference signals collected by the receiving coil in the at least one first time window based on the first interference signals. The system may further determine imaging signals collected by the receiving coil in the at least one first time window by correcting the initial signals based on the second interference signals.

In another aspect of the present disclosure, a system is provided. The system may include an obtaining module, an interference signal determination module, and a correction module. The obtaining module may be configured to obtain initial signals and first interference signals collected in at least one first time window during a Magnetic Resonance (MR) scan of a target subject. The nuclei in the target subject may be in an excited state in the at least one first time window. The initial signals may be collected by a receiving coil of a Magnetic Resonance Imaging (MRI) device. The first interference signals may be collected by an interference signal acquisition device. The interference determination module may be configured to determine second interference signals collected by the receiving coil in the at least one first time window based on the first interference signals. The correction module may be configured to determine imaging signals collected by the receiving coil in the at least one first time window by correcting the initial signals based on the second interference signals.

In another aspect, a non-transitory computer readable medium is provided. The medium may include executable instructions that, when executed by at least one processor, direct the at least one processor to perform a method. The method may include obtaining initial signals and first interference signals collected in at least one first time window during a Magnetic Resonance (MR) scan of a target subject. The nuclei in the target subject may be in an excited state in the at least one first time window. The initial signals may be collected by a receiving coil of a Magnetic Resonance Imaging (MRI) device. The first interference signals may be collected by an interference signal acquisition device. The method may also include determining second interference signals collected by the receiving coil in the at least one first time window based on the first interference signals. The method may further include determining imaging signals collected by the receiving coil in the at least one first time window by correcting the initial signals based on the second interference signals.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary signal collection and processing according to some embodiments of the present disclosure;

FIG. 2 is a schematic diagram illustrating an exemplary interference signal acquisition device arranged on an MRI device according to some embodiments of the present disclosure;

FIG. 3 is a schematic diagram illustrating an exemplary coil assembly according to some embodiments of the present disclosure;

FIG. 4 is a schematic diagram illustrating an exemplary application scenario of the coil assembly 300 according to some embodiments of the present disclosure;

FIG. 5 is a schematic diagram illustrating an exemplary circuit diagram of an interference signal acquisition device according to some embodiments of the present disclosure;

FIG. 6 is a schematic diagram illustrating an exemplary equivalent detuned circuit according to some embodiments of the present disclosure;

FIG. 7 is a schematic diagram illustrating an exemplary coil assembly under a natural expansion state according to some embodiments of the present disclosure;

FIG. 8 is a schematic diagram illustrating an exemplary arrangement of an acquisition unit and a housing according to some embodiments of the present disclosure;

FIG. 9 is a schematic diagram illustrating an exemplary acquisition unit according to some embodiments of the present disclosure;

FIGS. 10A and 10B are schematic diagrams illustrating an exemplary coil assembly according to some embodiments of the present disclosure;

FIG. 11 is a schematic diagram illustrating an exemplary enlarged view of a mounting base and an acquisition coil of an acquisition unit according to some embodiments of the present disclosure;

FIGS. 12 and 13 are schematic diagrams illustrating an exemplary application scenario of a coil assembly according to some embodiments of the present disclosure;

FIG. 14 is a schematic diagram illustrating an exemplary sectional view of a part of an MRI device along an axial direction of the MRI device according to some embodiments of the present disclosure;

FIG. 15 is a schematic diagram illustrating an exemplary radial view of a support cylinder according to some embodiments of the present disclosure;

FIG. 16 is a schematic diagram illustrating an exemplary sectional view of a part of the MRI device along the axial direction thereof according to some embodiments of the present disclosure;

FIG. 17 is a schematic diagram illustrating an exemplary couch according to some embodiments of the present disclosure;

FIG. 18 is a schematic diagram illustrating an exemplary processing device according to some embodiments of the present disclosure;

FIG. 19 is a flowchart illustrating an exemplary process for signal correction according to some embodiments of the present disclosure;

FIG. 20 is a schematic diagram illustrating an exemplary process of correcting EMI signals according to some embodiments of the present disclosure;

FIG. 21 is a schematic diagram illustrating an exemplary process for determining an interference signal determination model according to some embodiments of the present disclosure;

FIG. 22 is a schematic diagram illustrating an exemplary process for determining second interference signals according to some embodiments of the present disclosure; and

FIG. 23 is a schematic diagram illustrating an exemplary process for determining second interference signals according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included in programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “imaging modality” or “modality” as used herein broadly refers to an imaging method or technology that gathers, generates, processes, and/or analyzes imaging information of a subject. The subject may include a biological subject and/or a non-biological subject. The biological subject may be a human being, an animal, a plant, or a portion thereof (e.g., a cell, a tissue, an organ, etc.). In some embodiments, the subject may be a man-made composition of organic and/or inorganic matters that are with or without life. The term “subject” or “object” are used interchangeably.

An aspect of the present disclosure relates to a method for interference signal collection and/or processing (e.g., correction). The method may be implemented by a computing device including a storage device and at least one processor. The method may include obtaining initial signals and first interference signals collected in at least one first time window during a Magnetic Resonance (MR) scan of a target subject. The nuclei in the target subject may be in an excited state in the at least one first time window. The initial signals may be collected by a receiving coil of a Magnetic Resonance Imaging (MRI) device, and the first interference signals may be collected by an interference signal acquisition device. The method may also include determining second interference signals collected by the receiving coil in the at least one first time window based on the first interference signals. The method may further include determining imaging signals collected by the receiving coil in the at least one first time window by correcting the initial signals based on the second interference signals.

According to some embodiments of the present disclosure, the target subject may be placed in the main magnet field and scanned by the MRI device. The nuclei in the target subject may be excited to acquire initial signals and the first interference signals at the same time window (i.e., the first time window). The interference signals collected by the receiving coil (i.e., the second interference signals) in the first time window may be predicted based on the first interference signals. The initial signals may be corrected based on the predicted interference signals to generate target signals (i.e., the imaging signals), thereby eliminating or reducing the interference of the interference signals on the quality of MR image(s) generated based on the target signals.

Another aspect of the present disclosure relates to a coil assembly for Magnetic Resonance Imaging (MRI). The coil assembly may include a housing forming an accommodation space configured to accommodate a target subject. The coil assembly may also include a receiving coil configured to collect initial signals during an MR scan of the target subject. The coil assembly may also include an interference signal acquisition device configured to collect interference signals during the MR scan, wherein the interference signals are used to correct the initial signals to determine imaging signals collected during the MR scan.

According to some embodiments of the present disclosure, during the MR scan, the initial signals collected by the receiving coil may include an electromagnetic interference component. By using the interference signal acquisition device, the interference signals may be collected individually for eliminating or removing the electromagnetic interference component from the initial signals, which improves the imaging effect and ensures the quality of MR images. In addition, the coil assembly in the present disclosure can work without a shielding cage, which reduces the appliance cost of the MRI device, thereby the coil assembly can be promoted and used widely (e.g., in economically underdeveloped areas).

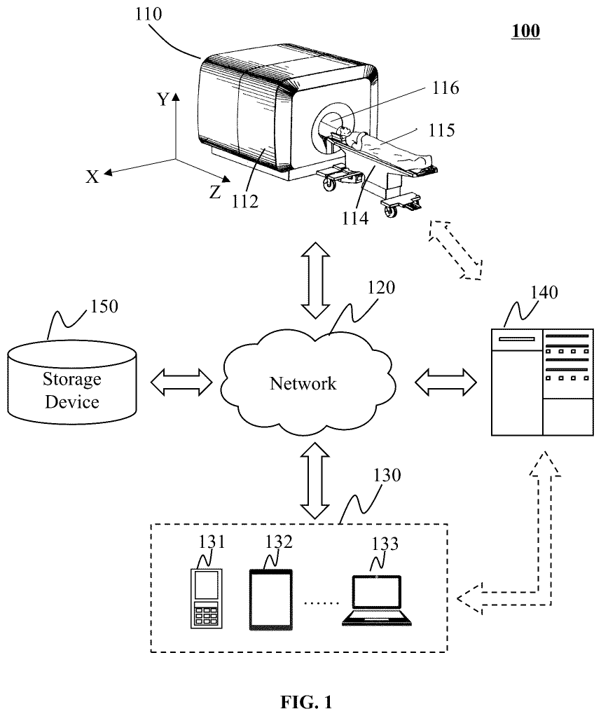

FIG. 1 is a schematic diagram illustrating an exemplary signal collection and processing system according to some embodiments of the present disclosure. The signal collection and processing system 100 may include a single-modality system (e.g., an MRI system) or a multi-modality system (e.g., an MRI-guided radiotherapy device). For illustration purposes, the signal collection and processing system 100 illustrated in FIG. 1 may include an MRI system. The MRI system may include an MRI device 110 such as an MRI scanner 110, a network 120, a terminal device 130, a processing device 140, and a storage device 150. The components of the signal collection and processing system 100 may be operably connected in one or more of various ways. Merely by way of example, as illustrated in FIG. 1, the MRI device 110 may be connected to the processing device 140 through the network 120.

In the present disclosure, the x-axis, the y-axis, and the z-axis shown in FIG. 1 may form an orthogonal coordinate system. The x-axis and the z-axis shown in FIG. 1 may be horizontal, and the y-axis may be vertical. As illustrated, the positive x-direction along the x-axis may be from the right side to the left side of the MRI device 110 seen from the direction facing the front of the MRI device 110; the positive y-direction along the y axis shown in FIG. 1 may be from the lower part to the upper part of the MRI device 110; the positive z-direction along the z-axis shown in FIG. 1 may refer to a direction in which a subject (e.g., a subject 115) is moved out of a detection region 116 of the MRI device 110.

The MRI device 110 may be configured to scan at least a part of the subject and acquire image data (or scan data) relating to the subject. The MRI device 110 may include magnets (not shown), coils (not shown), a gantry 112, a couch 114, etc. The magnets of the MRI device 110 may include a main magnet (e.g., a resistive magnet, a superconductive magnet, or a permanent magnet). The main magnet may form a bore (e.g., including a detection region 116) with an axis parallel to the z-direction as illustrated in FIG. 1, and surround the subject that is moved into or positioned along the z-direction within the detection region 116. The main magnet may be configured to generate a main magnetic field (B0 field). The main magnet may also control the homogeneity of the generated main magnetic field. The coils may include gradient coils, radio frequency (RF) coils, etc. The gradient coils may be located inside the bore formed by the main magnet. The gradient coils may be surrounded by the main magnet around the z-direction, and be closer to the subject than the main magnet. The gradient coils may be configured to generate a gradient magnetic field. The gradient magnetic field may be superimposed on the main magnetic field generated by the main magnet and distort the main magnetic field so that the magnetic orientations of the protons of the subject may vary as a function of their positions inside the gradient magnetic field, thereby encoding spatial information into MR signals generated by the region of the subject being imaged. The gradient coils may include an x gradient coil, a y gradient coil, and a z gradient coil. The x gradient coil, the y gradient coil, and the z gradient coil may be configured to generate gradient fields in the x-direction, the y-direction, and the z-direction, respectively, such that the MR signals can be spatially positioned. The RF coils may be located in the detection region 116 and serve as transmitters, receivers, or both. That is, the detection region 116 may be formed by the main magnet, the gradient coils, and the RF coils.

When used as transmitters, the RF coils may generate RF signals for providing a magnetic field that is utilized to excite MR signals related to the region of the subject being imaged. When used as receivers, the RF coils may also be referred to as a receiving coil responsible for detecting MR signals (e.g., echoes). For example, the RF coils may include volume transmitting coils (VTCs), local acquisition coils (e.g., surface coils), or the like, or any combination thereof, for detecting MR signals. The surface coils may be located closer to the region being imaged than the VTCs. The VTCs and/or the surface coils may include a birdcage coil, a solenoid coil, a saddle coil, a Helmholtz coil, an array coil, a loop coil, etc. The surface coils may include different specialized coils (e.g., head coil(s), spin coil(s), body surface coil(s), neck coil(s), limb coil(s), etc.) for different parts of the subject. The surface coils may be detachably arranged on the MRI device 110 or the subject.

The gantry 112 may be configured to support the magnets (e.g., the main magnet), the coils (e.g., the gradient coils and/or the RF coils), etc. The gantry 112 may surround, along the z-direction, the subject that is moved into or located within the detection region 116. The couch 114 may be configured to support the subject. Accordingly, the position of the subject within the detection region 116 may be adjusted by adjusting the couch 114. Merely by way of example, the couch 114 may move the subject into the detection region 116 along the z-direction in FIG. 1. In some embodiments, during the MR scan, a spinal coil may be used under the subject for scanning the back of the subject. More descriptions regarding the MRI device 110 can be found elsewhere in the present disclosure.

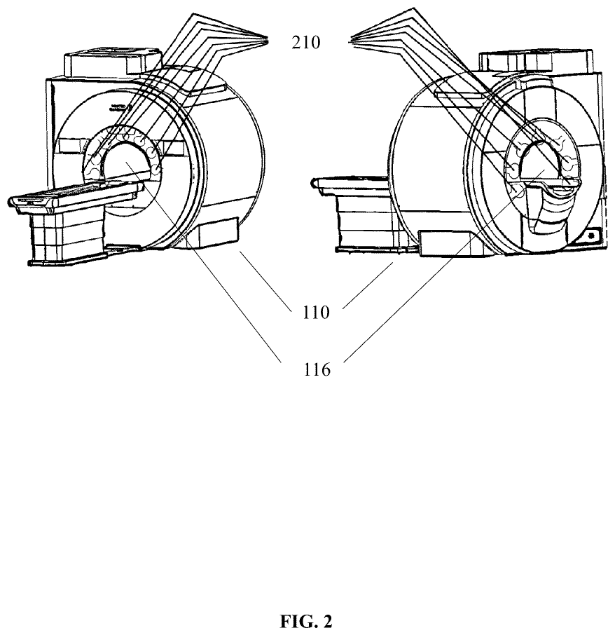

In some embodiments, when used as receivers, the RF coils may collect interference signals in the MR scan. In other words, initial signals collected by the RF coil in the MR scan may include pure MR signals (i.e., imaging signals) and also interference signals that need to be removed. In some embodiments, the MR device 110 may further include an interference signal acquisition device used to collect interference signals during the MR scan, wherein the interference signals are used to correct the initial signals to determine imaging signals collected during the MR scan. For example, the interference signal acquisition device and the receiving coil may be integrated as a coil assembly (e.g., a coil assembly 300 or 1000). More descriptions regarding the interference signal acquisition device and/or the receiving coil can be found elsewhere in the present disclosure (e.g., FIGS. 2-17 and relevant descriptions thereof).

The network 120 may include any suitable network that can facilitate the exchange of information and/or data for the signal collection and processing system 100. In some embodiments, one or more components of the signal collection and processing system 100 (e.g., the MRI device 110, the terminal device 130, the processing device 140, or the storage device 150) may communicate information and/or data with one or more other components of the signal collection and processing system 100 via the network 120. In some embodiments, the network 120 may be any type of wired or wireless network, or a combination thereof.

The terminal device 130 may include a mobile device 131, a tablet computer 132, a laptop computer 133, or the like, or any combination thereof. In some embodiments, the mobile device 131 may include a smart home device, a wearable device, a smart mobile device, a virtual reality device, an augmented reality device, or the like, or any combination thereof. In some embodiments, the terminal device 130 may remotely operate the MRI device 110 and/or the processing device 140. In some embodiments, the terminal device 130 may operate the MRI device 110 and/or the processing device 140 via a wireless connection. In some embodiments, the terminal device 130 may receive information and/or instructions inputted by a user, and send the received information and/or instructions to the MRI device 110 or the processing device 140 via the network 120. In some embodiments, the terminal device 130 may receive data and/or information from the processing device 140. In some embodiments, the terminal device 130 may be part of the processing device 140. In some embodiments, the terminal device 130 may be omitted.

The processing device 140 may process data and/or information obtained from the MRI device 110, the terminal device 130, and/or the storage device 150. For example, the processing device 140 may remove interference signals from initial signals collected by a receiving coil of the MRI device 110 in an MR scan. As another example, the processing device 140 may generate an interference signal determination model.

In some embodiments, the processing device 140 may be a single server, or a server group. The server group may be centralized or distributed. In some embodiments, the processing device 140 may be local or remote. In some embodiments, the processing device 140 may be implemented on a cloud platform. For example, a cloud platform may include a private cloud, a public cloud, a hybrid cloud, a community cloud, a distributed cloud, an inter-cloud, and a multi-cloud, or the like, or any combination thereof.

The storage device 150 may store data and/or instructions. In some embodiments, the storage device 150 may store data obtained from the MRI device 110, the terminal device 130, and/or the processing device 140. In some embodiments, the storage device 150 may store data and/or instructions that the processing device 140 may execute or use to perform exemplary methods described in the present disclosure. In some embodiments, the storage device 150 may include a mass storage device, a removable storage device, a volatile read-and-write memory, a read-only memory (ROM), or the like, or any combination thereof. In some embodiments, the storage device 150 may be implemented on a cloud platform.

In some embodiments, the storage device 150 may be connected to the network 120 to communicate with one or more components of the signal collection and processing system 100 (e.g., the MRI device 110, the processing device 140, the terminal device 130, etc.). One or more components of the signal collection and processing system 100 may access the data or instructions stored in the storage device 150 via the network 120. In some embodiments, the storage device 150 may be part of the processing device 140 or may be independent and directly or indirectly connected to the processing device 140.

It should be noted that the above description of the signal collection and processing system 100 is merely provided for the purposes of illustration, and not intended to limit the scope of the present disclosure. For persons having ordinary skills in the art, multiple variations and modifications may be made under the teachings of the present disclosure. For example, the signal collection and processing system 100 may include one or more additional components and/or one or more components of the signal collection and processing system 100 described above may be omitted. Additionally or alternatively, two or more components of the signal collection and processing system 100 may be integrated into a single component. A component of the signal collection and processing system 100 may be implemented on two or more sub-components.

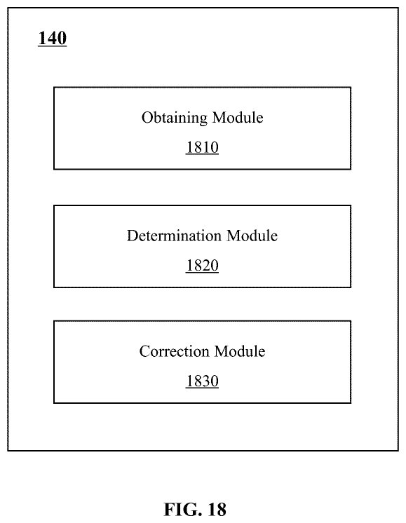

FIG. 2 is a schematic diagram illustrating an exemplary interference signal acquisition device arranged on an MRI device according to some embodiments of the present disclosure. As described in connection with FIG. 1, during the imaging process, electromagnetic waves generated by external interference sources may affect the homogeneity of the main magnetic field generated by the main magnet of the MRI device 110, which results in MR images of the subject 115 with low signal to noise ratio (SNR) and/or artifacts. To obtain MRI images of the subject 115 with high image quality, initial signals acquired by the MRI device 110 may need to be processed for correcting the interferences and determining pure imaging signals (i.e., MR signals). For example, the subject 115 may be placed in the detection region 116 within the main magnetic field. In the working state, the MRI device 110 may apply a scanning sequence (i.e., perform an MR scan) to excite the nuclei in the subject 115 and obtain initial signals and first interference signals. The initial signals and the first interference signals may be collected during the same time window (e.g., a first time window) at which the scanning sequence is performed, and stored correspondingly. The first interference signals may be used to determine predicted interference signals collected by receiving coil (also referred to as second interference signals) in the first time window for correcting the initial signals to obtain target imaging signals, more descriptions of which can be found elsewhere in the present disclosure (e.g., FIGS. 18-23 and the descriptions thereof).

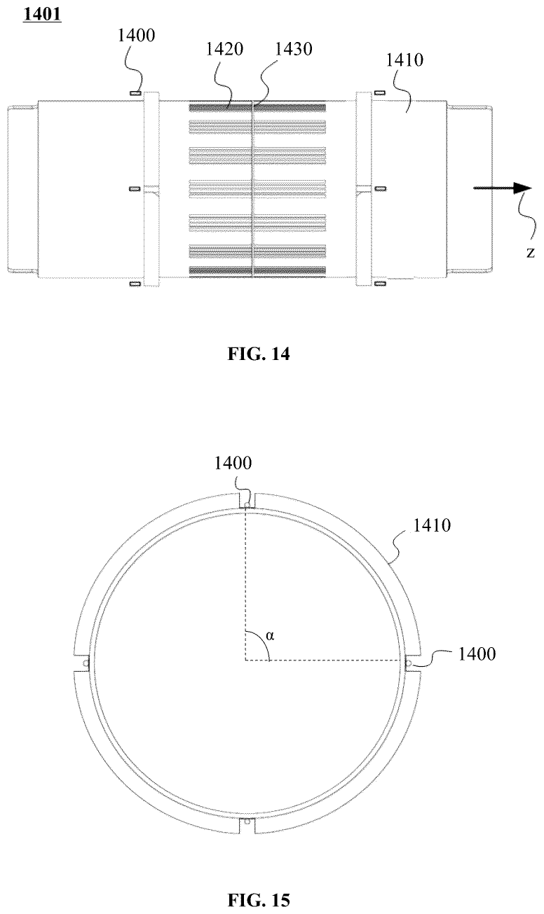

In some embodiments, the MRI device 110 may further include an interference signal acquisition device 210 configured to collect electromagnetic interference (EMI) signals (e.g., the first interference signals) during the MR scan. In some embodiments, the interference signal acquisition device 210 may include one or more acquisition units each of which includes an acquisition coil. For example, as shown in FIG. 2, the interference signal acquisition device 210 may include a plurality of acquisition coils. The plurality of acquisition coils may be arranged on two ends of the detection region 116 (e.g., at two ends of the gantry 112) at which a shielding coil can not achieve interference signal shielding. In some embodiments, the interference signal acquisition device 210 may be arranged outside the detection region 116, and a receiving coil (e.g., the RF coil such as a surface coil) of the MRI device may be arranged within the detection region 116 of the MRI device 110, more descriptions of which can be found elsewhere in the present disclosure (e.g., FIGS. 14-17 and the relevant descriptions thereof). In some embodiments, the interference signal acquisition device 210 and the receiving coil may be integrated via a housing to form a coil assembly of the MRI device 210. When the receiving coil is a surface coil, according to the type of the surface coil, the coil assembly may include an abdomen coil assembly, a head coil assembly, a knee coil, a neck coil assembly, or the like, or any combination thereof. More descriptions regarding the coil assembly and the components thereof can be found elsewhere in the present disclosure (e.g., FIGS. 3-13 and the relevant descriptions thereof).

By arranging the plurality of acquisition coils at two ends of the detection region 116 of the MRI device 110 for shielding or correcting the interference signals in the surrounding environment, the overall design of the MRI device may be not changed, and the plurality of acquisition coils may be controlled to collect the interference signals by an additional digital switch, which can reduce design and layout costs of the MRI device 110. In addition, the collected interference signals may be used for correcting the initial signals, which can accurately remove or correct the interference signals that enter the detection region 116 from the two ends of the detection region 116, thereby achieving accurate shielding of the interference signals entered the detection region 116 and improving the imaging quality of the MRI device 110.

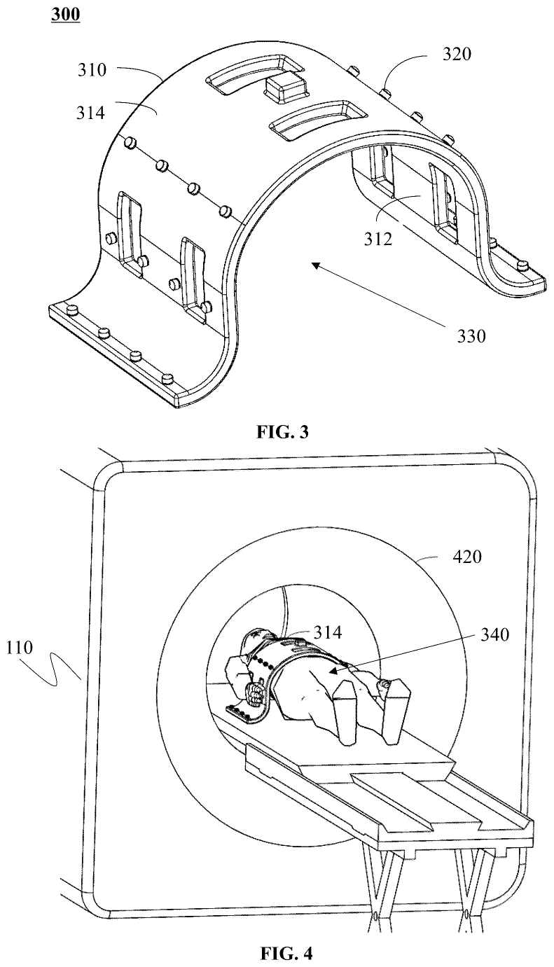

FIG. 3 is a schematic diagram illustrating an exemplary coil assembly according to some embodiments of the present disclosure. The coil assembly 300 may be an abdomen coil assembly for an abdomen scan. As shown in FIG. 3, the coil assembly 300 may include a housing 310, a receiving coil (not shown), and an interference signal acquisition device (including one or more acquisition units 320). During an MR scan, the coil assembly 300 may be located within a main magnetic field environment generated by an MRI device (e.g., the MRI device 110 as shown in FIG. 4).

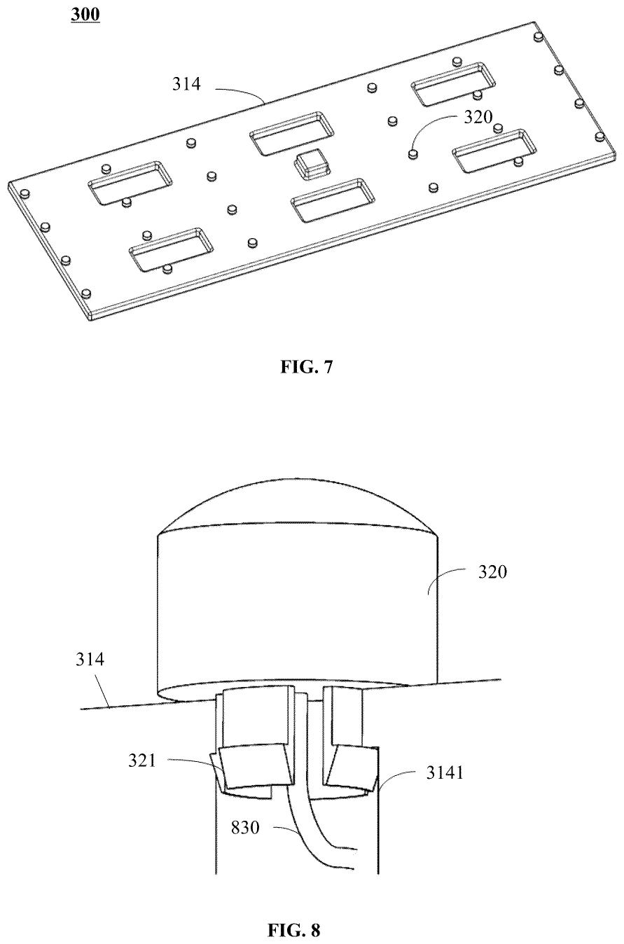

The housing 310 may form an accommodation space 310 configured to accommodate a target subject or a portion thereof (e.g., the abdomen of a target subject 340 as shown in FIG. 4). In some embodiments, the housing 310 may include a first external surface 312 facing the target subject and a second external surface 314 opposite to the first external surface 312. More descriptions regarding the housing 310 can be found elsewhere in the present disclosure (e.g., FIG. 7 and relevant description thereof).

The receiving coil may be configured to collect initial signals during an MR scan of the target subject. In some embodiments, the receiving coil may be arranged within the housing 310. For example, the receiving coil may be arranged within the housing 310 between the first external surface 312 and the second external surface 314 of the housing 310. In some embodiments, the receiving coil may include a plurality of coil units. For example, the receiving coil may include an RF coil that includes a plurality of coil units. The plurality of coil units may be arranged within the housing 310 between the first external surface 312 and the second external surface 314 of the housing 310. In some embodiments, the plurality of coil units may be arranged on a flat surface or a curved surface coinciding with the housing 310. For example, when the coil assembly 300 is used for scanning an abdomen and arranged surrounding the abdomen of the target subject 340, the first external surface 312 may be in contact with the peripheral side of the abdomen of the target subject 340, and the plurality of coil units may be correspondingly arranged surrounding to the peripheral side of the abdomen of the target subject 340. MR signals of the abdomen of the target subject may be collected by the plurality of coil units.

The interference signal acquisition device may be configured to collect interference signals during the MR scan. The interference signals may be used to correct the initial signals to determine imaging signals collected during the MR scan. The interference signal acquisition device may include the one or more acquisition units 320. In some embodiments, the one or more acquisition units 320 of the interference signal acquisition device may be arranged in parallel and supported by the second external surface 314 of the housing 310.

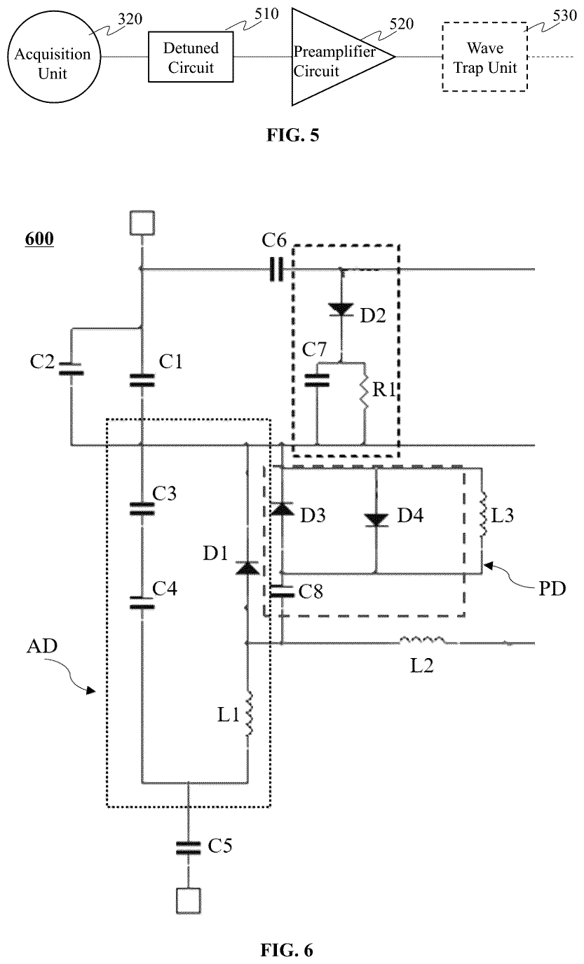

In some embodiments, each of the one or more acquisition units 320 may include a probe or an acquisition coil. For example, an acquisition unit 320 may include an electromagnetic interference (EMI) probe. The EMI probe may be configured to collect interference signals in the main magnetic field environment wherein the coil assembly 300 is located. The interference signals may include an electromagnetic interference signal, a direct current interference caused by a large electrical device (e.g., a tram, a subway, etc.), an alternating current interference caused by an alternating current electric field outside the MR device 110, or the like, or any combination thereof. The EMI probe may also be not able to detect MR signals or can only receive weak MR signals, which helps the removal of interference components in the MR signals collected by the coil assembly 300. More descriptions regarding the acquisition units 340 of the interference signal acquisition device can be found elsewhere in the present disclosure (e.g., FIG. 5 and the relevant descriptions thereof).

FIG. 4 is a schematic diagram illustrating an exemplary application scenario of the coil assembly 300 according to some embodiments of the present disclosure. As shown in FIG. 4, the main magnetic field environment where the coil assembly 300 is located may be a main magnetic field environment generated by the main magnet of the MRI device 110, and the target subject 340 may be placed in the main magnetic field environment before being scanned. In some embodiments, the MRI device 110 may include a volume transmit coil (VTC) 420 for emitting RF pulses. If the VTC 420 is in a state of emitting RF pulses, the receiving coil of the coil assembly 300 may be adjusted to a detuned state; when the MR scan is performed, the receiving coil may collect initial signals (including imaging signals and interference signals) and the acquisition units 320 of the interference signal acquisition device may collect interference signals. If the VTC 420 is in a state of not emitting RF pulses and the MR scan is not performed, the receiving coil and the acquisition units 320 of the interference signal acquisition device may simultaneously collect interference signals to accurately determine interference signals of the main magnetic field where the coil assembly 300 is located.

In some embodiments, an acquisition unit 320 of the interference signal acquisition device may include a signal detection interface/end. The signal detection end may include an acquisition coil. A surface of the acquisition coil and a surface of each coil unit of the receiving coil may form a preset angle.

In some embodiments, the preset angle may be greater than 0 degrees and less than 180 degrees. If the VTC 420 is in a state of emitting RF pulses towards the target subject 340 located in the main magnetic field environment, the precession flip of the proton group in the target subject 340 may generate a magnetization vector Mxy in the x-y plane. If the magnetization vector Mxy is directed in the same direction as the acquisition direction of the receiving coil, the receiving coil can receive maximum MR signals. For example, the preset angle may be set to 90 degrees, that is, the acquisition direction of the acquisition coil of the acquisition unit 320 may be set to be orthogonal to the acquisition direction of the coil unit of the receiving coil. If the direction of the magnetic field that causes the precession of the proton group is orthogonal to the direction of the acquisition direction of the acquisition coil and parallel to the direction of the main magnetic field (B0 field) generated by the MRI device 110, the receiving coil can receive almost no MR signals. By setting the acquisition direction of the acquisition coil to be orthogonal to the acquisition direction of the coil unit, the coil unit can collect the maximum MR signals, while the acquisition coil of the acquisition unit 320 can receive almost no MR signals. In some embodiments, the acquisition direction of the acquisition coil may be set to be parallel to the direction of the main magnetic field (B0 field) generated by the MRI device 110, e.g., by setting a placement direction of the acquisition unit.

In some embodiments, a size of the acquisition coil may be smaller than a size of each coil unit of the receiving coil, such that a penetration depth of the acquisition unit 320 (e.g., the probe) may be relatively short. By setting the acquisition unit 320 on the second external surface 314 of the housing 310, the acquisition unit 320 may be farther away from the target subject 340 than the receiving coil within the housing 310. Since MR signals decay in signal transmission, the acquisition units 320 farther away from the target subject 340 can receive almost no MR signals, thereby causing the acquisition units 320 to receive interference signals of the main magnetic field environment more accurately.

FIG. 5 is a schematic diagram illustrating an exemplary circuit diagram of an interference signal acquisition device according to some embodiments of the present disclosure. As shown in FIG. 5, besides the acquisition unit 320, the interference signal acquisition device may further include a detuned circuit 510, a preamplifier circuit 520, and a wave trap unit 530.

The detuned circuit 510 may be electrically connected to the acquisition unit 320. For example, an input interface/end of the detuned circuit 510 may be electrically connected with an output interface/end of the acquisition unit 320. In some embodiments, the detuned circuit 510 may be configured to adjust the acquisition unit 320 (e.g., an acquisition coil thereof) to a detuned state or a resonant state. That is, the acquisition unit 320 can switch between the detuned state and the resonant state. For example, if the VTC 420 of the MRI device 110 is in a state of emitting RF pulses, the detuned circuit 510 may be configured to adjust the acquisition unit 320 (e.g., the acquisition coil thereof) to a detuned state, thereby ensuring a signal receiving circuit (e.g., the preamplifier circuit 520) connected subsequently to the detuned circuit 510 may not be damaged. More descriptions regarding the detuned circuit 510 can be found elsewhere in the present disclosure (e.g., FIG. 6 and the description thereof).

The preamplifier circuit 520 may be electrically connected to the detuned circuit 510 and the wave trap unit 530. For example, an input interface/end of the preamplifier circuit 520 may be electrically connected with an output interface/end of the detuned circuit 510. As another example, an output interface/end of the preamplifier circuit 520 may be electrically connected with an input interface/end of the wave trap unit 530. In some embodiments, the preamplifier circuit 520 may be configured to amplify the interference signals collected by the acquisition unit 320. For example, the preamplifier circuit 520 may include a low noise amplifier (LNA).

The wave trap unit 530 (also referred to as a trap 530) may be configured to ensure the effective transmission of the interference signals or the amplified interference signals. In some embodiments, there may include more than one trap 530 (e.g., a plurality of traps 530). The plurality of traps 530 may be arranged at intervals (e.g., per 30 cm).

FIG. 6 is a schematic diagram illustrating an exemplary equivalent detuned circuit according to some embodiments of the present disclosure. The equivalent detuned circuit 600 may be an equivalent circuit of the detuned circuit 510 as described in FIG. 5.

The detuned circuit 600 may include an active detuning (AD) circuit and a passive detuning (PD) circuit. Capacitor C1 and capacitor C2 are connected in parallel. Capacitor C5 is a series frequency modulation capacitor. Capacitor C1 and capacitor C2 are connected to one end of an acquisition unit 320 (e.g., an EMI probe), and capacitor C5 is connected to the other end of the acquisition unit 320. An active detuning (AD) circuit is connected between capacitor C5 and a parallel matching circuit formed by capacitor C1 and capacitor C2. The AD circuit includes capacitors C3 and C4 that are connected in series, and diode D1 and inductor L1 that are connected in series. The cathode of diode D1 is connected to one end of capacitor C3, the anode of diode D1 is connected to one end of inductor L1, and inductor L1 is connected to a diode at one end of capacitor C4. The PD circuit includes diode D3, diode D4 that is connected to diode D3 in parallel reversely, and capacitor C8. One end of capacitor C8 is connected in series with each of diode D3 and diode D4, the other end of capacitor C8 is connected to the anode of diode D1, and the cathode of diode D3 is connected to the cathode of diode D1. Further, diode D2, resistor R1, capacitor C6 form a protection circuit for the preamplifier circuit 520.

For example, the AD circuit may control connection and disconnection of diode D1 by applying a direct current (DC) signal to inductor L2. When diode D1 is connected (i.e., turned on), capacitor C3, capacitor C4, and inductor L1 may form a parallel resonant circuit, which corresponds to a high impedance state. In such cases, the acquisition unit 320 may be regarded as being disconnected in the working frequency of the MRI device 110 (resonant frequency of the coil assembly 300), therefore, the acquisition unit 320 can not receive imaging signals (i.e., MR signals). The PD circuit may control connections and disconnections of diode D3 and diode D4 by RF signals (i.e., AC signals induced by the receiving coil through an RF field when the MRI device 110 is working (mainly referring to emitting signals)). When the active detune is off, the acquisition unit 320 may detect/sense the RF signals and generate alternating current (AD) signals. The alternating current may pass parallel matching capacitors C1 and C2 and make diodes in two revered directions to be electronically connected. Capacitor C3, capacitor C4, and inductor L1 may form a parallel resonant circuit, which corresponds to a high impedance state. In such cases, the acquisition unit 320 may be regarded as being disconnected in the working frequency of the MRI device 110 (resonant frequency of the coil assembly 300).

FIG. 7 is a schematic diagram illustrating the coil assembly 300 under a natural expansion state according to some embodiments of the present disclosure. FIG. 7 shows an exemplary shape of the housing 310 of the coil assembly 300 under the natural expansion state.

The housing 310 may be a flexible housing used to accommodate flexible circuits, such that the coil assembly 300 can coincide with the contour of the abdomen of the target subject 340. For example, the housing 310 may be made of fiber, a fiber product, animal fur, felt cloth, rubber, plastic, or the like, or any combination thereof. For instance, the housing 310 may be made of fiber, fabric, or a compound thereof. Each coil unit of the receiving coil may be flexibly encapsulated by the flexible housing 310. The flexible housing 310 may not affect the softness of the receiving coil, such that the coil assembly 300 can fit well to the target subject 340, thereby improving the fit degree between the coil assembly 300 and the target subject 340.

The coil unit(s) of the receiving coil may be made of copper, coaxial line, elastic metal wire, liquid metal wire, or the like, or any combination thereof. In some embodiments, a coil unit of the receiving coil may be made of an elastic metal wire or a liquid metal wire formed by an elastic material or a liquid metal. The elastic metal wire or the liquid metal wire composed of non-magnetic materials can make the coil unit maintain good elasticity, better fit the target subject 340, improve RF signal strength, and improve imaging quality. In addition, as the resonant frequency is broadband, the reliability of RF signals may be guaranteed even under the maximum deformation of the flexible housing 310. The insensitive structure of the receiving coil may minimize the sensitivity of signal acquisition when the coil unit of the receiving coil is deformed. In some embodiments, the coil unit may include a flexible insulating support and a flexible conductor. The insulating support may be bent along a preset direction, and the flexible conductor may be combined with the insulating support and extend along a direction consistent with the axial direction of the insulating support. The insulating support may include a deformable component and be set as a solid structure. The flexible conductor may include a metal strip, a metal tube, a metal braided layer, or the like, or any combination thereof. The flexible conductor may be set as a hollow structure, and the insulating support and the flexible conductor may form a coaxial structure.

In some embodiments, a metal braided layer may be formed by a plurality of conductive wires and the metal braided layer may cover the external side of the insulating support. In some embodiments, the deformable component of the insulating support may include a fibrous material, a composite material, a copolymer material, etc., such that the insulating support has good bending properties as well as good insulation properties. The conductive wire forming the metal braided layer may include gold wires, copper wires, aluminum wires, silver wires, carbon fiber conductive wires, etc. The insulating support and the flexible conductor may form a coaxial structure. For example, the insulating support may be set as the core of the coil unit, and the flexible conductor may be set as the outer layer of the coil unit, which can reduce the thickness of the flexible conductor. In some embodiments, the coil unit may include an inner conductor and an outer conductor. For example, the inner conductor may be arranged in parallel with the outer conductor, or the inner conductor and outer conductor may be arranged coaxially to form a coaxial structure. As another example, the outer conductor may be winded in a spiral shape around the inner conductor, and the inner conductor may be bent in a preset direction to form a loop. An insulating dielectric material may be arranged between the inner conductor and the outer conductor.

In some embodiments, the second external surface 314 of the housing 310 may be provided with one or more slots (not shown in FIG. 7). The slots may be arranged in columns and/or rows. Each of the one or more acquisition units 320 may be provided with a buckle (not shown in FIG. 7) at a bottom of the acquisition unit 320. The buckle may correspond to one of the one or more slots and can be buckled in the corresponding slot. The acquisition unit 320 of the interference signal acquisition device may include a connection wire via which the output end of the acquisition unit 320 (e.g., an output end of an acquisition coil thereof) may be connected with an input end of the detuned circuit 510. The buckle may be provided with a through hole through which the connection wire of the interference signal acquisition device is passed to connect the corresponding acquisition coil with the detuned circuit 510.

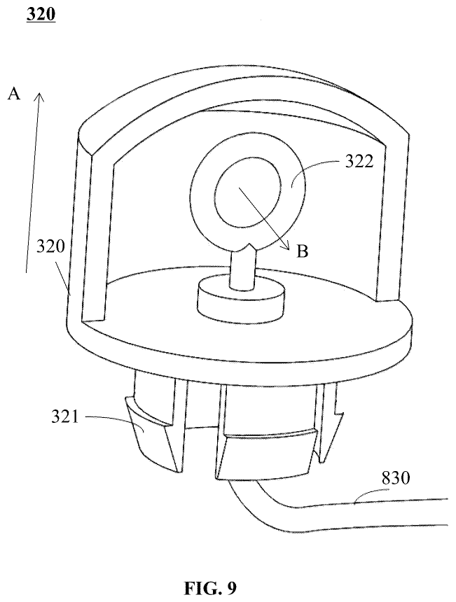

For example, as shown in FIG. 8, the second external surface 314 of the housing 310 may be provided with a slot 3141. The acquisition unit 320 may be provided with a buckle 321 at the bottom of the acquisition unit. The acquisition unit 320 may be fixed on the second external surface 314 via a fit between the slot 3141 and the buckle 321. When the buckle 321 is pressed and buckled in the slot 3141, the rotation and translation of the acquisition unit 320 may be limited. A connection wire 830 may pass through a through hole of the buckle 321 to connect the output end of the acquisition unit 320 and the input end of the detuned circuit 510.

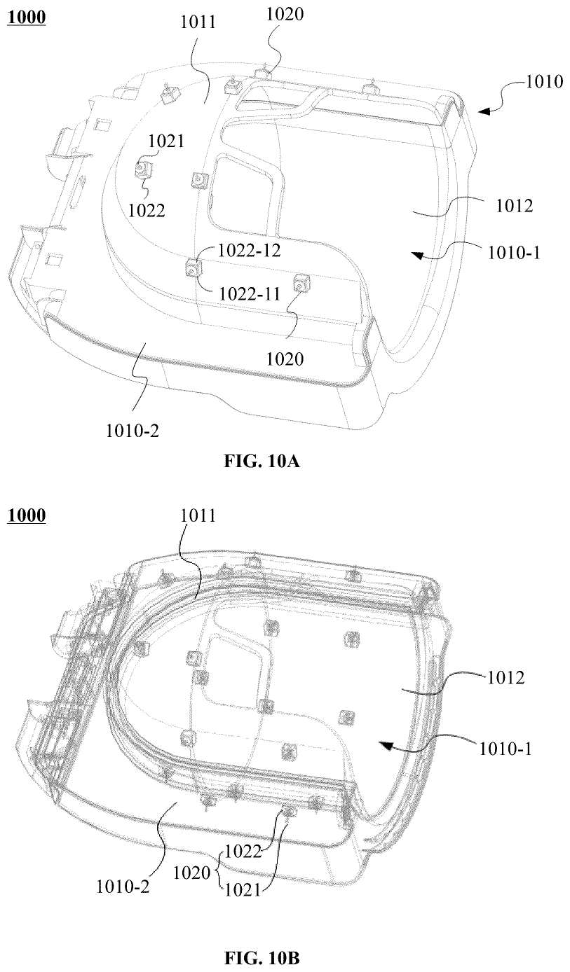

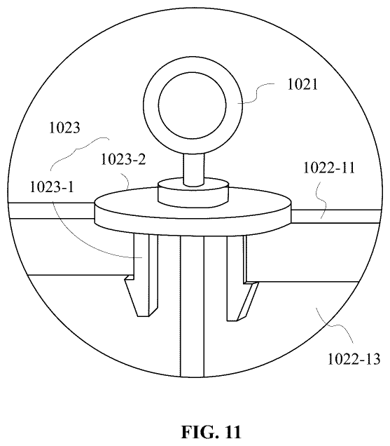

As shown in FIG. 9, in some embodiments, the acquisition unit 320 may include an acquisition coil (e.g., a toroidal inductor) 322 configured to collect electromagnetic interference signals along a central axis of the acquisition coil 322. For example, the axial direction (as illustrated by arrow A perpendicular to a bottom surface of the acquisition unit 320) of the acquisition unit 320 may be perpendicular to the second external surface 314, the axial direction (as illustrated by arrow B perpendicular to a surface where the acquisition coil 322 is located on) of the acquisition coil 322 may be perpendicular to the acquisition unit 320. In some embodiments, the axial direction of the acquisition coil 322 may be determined based on a direction of the main magnetic field of the MRI device 110 that is used to scan the target subject 340. For example, the axial direction of the acquisition unit 320 may be perpendicular to the direction of the main magnetic field. When the coil assembly 300 contacts the target subject 340 for scanning, the acquisition direction of the acquisition coil 322 may be consistent with the axial direction of the acquisition coil 322, which is the acquisition direction of the acquisition unit 320. The acquisition direction of the acquisition unit 320 may be parallel to the main magnetic field B0, and orthogonal to the precession direction of the proton group, such that the acquisition unit 320 can collect as useful interference signals as possible and collect MR signals as little as possible. The acquisition unit 321 may be similar to an acquisition unit 1020 described else elsewhere in the present disclosure (e.g., FIGS. 10A, 10B, and 11 and relevant descriptions thereof).

In some embodiments, the receiving coil (e.g., an RF coil) including a plurality of coil units. Each of the plurality of coil units may include a conductor ring whose axial direction forms a preset angle with the main magnetic field of the MRI device 110. That is, the axial direction of the conductor ring of the coil unit may be not parallel to the direction of the main magnetic field of the MRI device 110. When the VTC 420 is in a state of emitting RF signals, the conductor ring of the coil unit may collect a magnetization vector Mxy in the x-y plane generated by the precession flip of the proton group, thereby achieving the collection of MR signals. The acquisition direction of the acquisition coil 322 may be parallel to the direction of the magnetic main field of the VTC 420. When the VTC 420 is in a state of emitting RF signals, the acquisition coil 322 may collect the magnetization vector Mxy in the x-y plane generated by the precession flip of the proton group as little as possible, such that the acquisition unit 320 can collect MR signals as little as possible and collect as useful interference signals as possible.

It should be noted that the descriptions of the coil assembly 300 and components thereof are merely provided for the purposes of illustration, and not intended to limit the scope of the present disclosure. For persons having ordinary skills in the art, multiple variations and modifications may be made under the teachings of the present disclosure. In some embodiments, the acquisition units 320 of the interference signal acquisition device may be integrated inside the housing 310.

FIGS. 10A and 10B are schematic diagrams illustrating an exemplary coil assembly according to some embodiments of the present disclosure. The coil assembly 1000 may be a head coil assembly for a head scan. As shown in FIG. 10A, similar to the coil assembly 300, the coil assembly may include a housing 1010, a receiving coil (not shown), and an interference signal acquisition device including one or more acquisition units 1020.

The housing 1010 may include two parts, e.g., a first housing 1011 (also referred to as an upper housing) and a second housing 1012 (also referred to as a lower housing). The first housing 1011 may cover a front end of the target subject (e.g., the head). The second housing 1012 may carry the first housing 1011 and support a back end of the target subject. As used herein, when the target subject is at a supine position, the front end of the target subject may refer to the front side of the head (i.e., the face) of the target subject, and the back end of the target subject may refer to the back side of the head of the target subject. The second housing 1012 and the first housing 1011 may form a first accommodation space 1010-1 configured to accommodate the target subject, e.g., configured to be worn on the head of the target subject. The second housing 1012 and the first housing 1011 may also form a second accommodation space 1010-2 configured to accommodate inner components of the coil assembly 1000.

Each of the acquisition units 1020 of the interference signal acquisition device may include an acquisition coil 1021 and a mounting base 1022. The acquisition coil 1021 may be configured to receive interference signals. The mounting base 1011 may be arranged on the first housing 1011 and connected with the acquisition coil 1021. The mounting base 1022 may be configured to support the acquisition coil 1021 on the first housing 1011 of the housing 1010. That is, the interference signal acquisition device may include one or more mounting bases 1022 configured to support the one or more acquisition coils 1021 on the first housing 1011 of the housing 1010. The interference signals collected by the one or more acquisition coils 1021 may be used for removing electromagnetic interference components in initial signals collected by the receiving coil of the coil assembly 1000.

As shown in FIG. 10B, at least part of the one or more acquisition coils 1021 may be arranged on an inner surface of the second housing 1012. As used herein, an inner surface of the second housing 1012 may refer to a surface of the second housing that is close to the first housing 1011. For example, the second housing 1012 may be provided with a mounting base 1022 at the inner surface of the second housing 1012, and the acquisition coil 1021 may be supported by the mounting base 1022 to extend toward the accommodation space 1010-2. When the target subject wears the coil assembly 1000, the acquisition coil(s) 1021 on the inner surface of the second housing 1012 may be arranged to correspond to the back end of the target subject, which can improve the acquisition range of interference signals of the acquisition unit 1020. The structures of the acquisition units 1021 may be the same as or similar to that of the acquisition unit 320 as described elsewhere in the present disclosure. It should be noted that the acquisition coil(s) 1021 on the second housing 1012 may be omitted, which is not limited herein.

Taking an acquisition unit 1020 arranged on the first housing 1011 as an example, a mounting base 1022 of the acquisition unit 1020 may be connected with the first housing 1011. As shown in FIG. 10A and FIG. 11, the mounting base 1022 may include a supporting part and a mounting part 1023. The supporting part may be connected with the first housing 1011. For example, the supporting part may include a top plate 1022-11 and a side wall 1022-12 connected with the top plate 1022-11. The top plate 1022-11 and the side wall 1022-12 may form an accommodation space 1022-13. A part of the supporting part opposite to the top plate 1022-11 may be connected with the first housing 1011 (e.g., by welding, an adhesive connection, or an integrated forming connection). The mounting part 1023 may be connected with the top plate 1022-11 of the supporting part. For example, the top plate 1022-11 may be provided with a mounting hole (which is similar to the slot as described in FIG. 8) for connecting the mounting part 1023.

In some embodiments, the mounting part 1023 may be connected with a part of the supporting part away from the first housing 1011 for limiting the position of the acquisition coil 1021, such that the acquisition coil 1021 can maintain a preset distance from the external surface of the first housing 1011. For example, the preset distance may be 8 cm˜2 cm (e.g., 1 cm, 1.2 cm, 1.4 cm, 1.6 cm, 1.8 cm, etc.0. With the preset distance, the MR signals collected by the acquisition coil 1021 may be weakened, and the coil assembly 1000 may be ensured to maintain a suitable size.

In some embodiments, the mounting part 1023 may include a first part 1023-1 (e.g., a buckle the same as or similar to the buckle 321 in FIG. 8) and a second part 1023-2. The first part 1023-1 may extend along an axial direction of the mounting hole of the top plate 1022-11 and be arranged in the mounting hole. An end of the first part 1023-1 located in the accommodation space 1022-13 may be provided with a position limiting part which abuts the top plate 1022-11 to avoid the mounting part 1023 from sliding up and out from the mounting hole. The second part 1023-2 may be located outside the accommodation space 1022-13, and extend towards the periphery of the mounting hole to avoid the mounting part 1023 from sliding down and into the mounting hole. The second part 1023-2 may also be provided with a through hole which is configured for passing through a transmission wire (e.g., which is similar to the connection wire 830 in FIG. 8) of the acquisition coil 1021, and the acquisition coil may be limited at a side of the second part 1023-2 away from the accommodation space 1022-13.

The receiving coil (e.g., an RF coil) may be arranged between the second housing 1012 and the first housing 1011. The receiving coil may be configured to collect MR signals during an MR scan of the target subject. For example, when the target subject wears the coil assembly 1000, the receiving coil may be arranged surrounding the head of the target subject for fully collecting the MR signals. The receiving coil may be similar to the receiving coil of the coil assembly 300.

In some embodiments, the receiving coil may also be configured to tune the interference signal acquisition device 1000 in a non-MR scanning stage (e.g., a second time window as described elsewhere in this disclosure) to collect interference signals. The receiving coil may include a tuned circuit that receives interference signals synchronously with the interference signal acquisition device 1000 in the non-MR scanning stage. The interference signals obtained by the receiving coil and interference signal acquisition device 1000 in the non-magnetic resonance scanning condition, as well as the interference signals obtained by interference signal acquisition device 1000 in the MR scanning signal acquisition stage (e.g., a first time window as described elsewhere in this disclosure), may be used as the basis for removing the electromagnetic interference components in the initial signals. In this way, the accuracy of the final MR images may be improved.

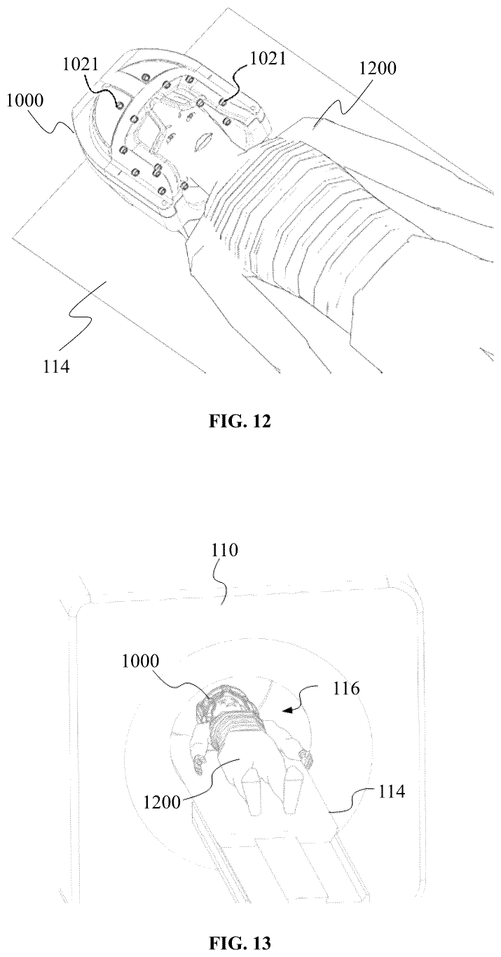

FIGS. 12 and 13 are schematic diagrams illustrating an exemplary application scenario of the coil assembly 1000 according to some embodiments of the present disclosure. As described in connection with FIGS. 10A and 10B, the coil assembly 1000 may include a plurality of acquisition coils 1021 and a plurality of mounting bases 1022. The plurality of mounting bases 1022 may be distributed on the first housing 1011 uniformly, such that the plurality of acquisition coils 1021 can be distributed uniformly. In the use state, the coil assembly 1000 may be worn on the head of a target subject 1200, and the plurality of acquisition coils 1021 may be distributed around the head of the target subject 1200 uniformly, such that the coil assembly 1000 can collect interference signals transmitted in different directions, thereby improving the accuracy of the collected interference signals. In addition, as the acquisition range of a single acquisition coil 1021 is limited, by setting the plurality of acquisition coils 1021, the acquisition range may be enlarged. As shown in FIGS. 12 and 13, during the MR scan, the target subject 200 wearing the coil assembly 1000 may be placed on the couch 114 and transmitted to the detection region 116 of the MRI device 110, such that the target subject 200 may be located in the main magnetic field generated by the MRI device 110.

In some embodiments, the axial direction of the acquisition coil 1021 may be arranged along a preset direction. The preset direction may be consistent with the magnetic direction of the main magnetic field generated by the MRI device 110. When the RF pulses (i.e., the RF signals) are emitted and the axial direction of the acquisition coil is orthogonal to the direction of a magnetic field generated by the precession flip of the proton group in the target subject 1200, the acquisition coil 1021 may not or substantially not receive MR signals. Accordingly, the axial direction of the acquisition coil 1021 may be set to be parallel to the magnetic direction of the main magnetic field, thereby ensuring that the acquisition coil 1021 receives weak MR signals.

In some embodiments, the diameter of the acquisition coil 1021 may be in a range of 1.5˜3.0 cm, such as 1.8 cm, 2 cm, 2.5 cm, or 2.8 cm. The diameter of the acquisition coil 1021 may be less than that of the receiving coil, which can reduce the penetration depth of the MR signals. As described in FIGS. 10A and 10B, the acquisition coil 1021 may be set to have a preset distance from the external surface of the first housing 1011, which further weakens the strength of the MR signals collected by the acquisition coil 1021.

In some embodiments, the interference signal acquisition device may further include a detuned circuit electrically connected with the acquisition coil 1021 and a signal processing circuit electrically connected with the detuned circuit. The detuned circuit may be configured to control the acquisition coil 1021 to be detuned at a stage of emitting RF signals, and control the acquisition coil 1021 to be tuned at a stage of collecting MR signals or a non-MR scanning stage. The detuned circuit may be the same as or similar to the detuned circuit 510 as described in FIG. 5, and an equivalent of the detuned circuit may be the same as or similar to that described in FIG. 6. The signal processing circuit may be configured to process the interference signals collected by the acquisition coil 1021 and/or transmit the processed signals to other devices (e.g., a display device) that work together with the coil assembly 1000. For example, the signal processing circuit may include an LNA, which is the same as or similar to the LNA 520 as described in FIG. 5.

In some embodiments, the interference signal acquisition device may also include a trap electrically connected to the output end of the signal processing circuit. The trap may be the same as or similar to the trap 530 as described in FIG. 5. In some embodiments, there may be a plurality of traps that are arranged at intervals according to a preset distance (e.g., 14 cm, 16 cm, 18 cm, etc.), such that signals at unnecessary frequencies may be filtered, thereby ensuring the accuracy of the obtained interference signals.

In some embodiments, as described in connection with the structure of the mounting base 1022, the detuned circuit may be connected with a transmission wire of the acquisition coil 1021; the detuned circuit, the signal processing circuit, and/or the trap(s) may be integrated into the accommodation space 1022-13 of the supporting part, thereby improving the integration level of the coil assembly 1000.

In some embodiments, the coil assembly 1000 may be fixed on the couch 114, such that the coil assembly 1000 can move in or out of the detection region 116 with the couch 114.

FIG. 14 is a schematic diagram illustrating an exemplary sectional view of a part of an MRI device 1401 along the axial direction (i.e., the z-direction) of the MRI device 1401 according to some embodiments of the present disclosure. As shown in FIG. 14, the MRI device 1401 may include a support cylinder 1410, leg antenna(s) 1420, and an end ring 1430. The support cylinder 1410 may form the bore including the detection region 116. The bore may be configured to support the leg antenna(s) 1420 and the end ring 1430. In some embodiments, there may be a plurality of leg antennas 1420 arranged on a surface of the support cylinder 1410. Each of the plurality of leg antennas 1420 may extend along an axial direction (i.e., the z-direction) of the support cylinder 1410. The plurality of leg antennas 1420 may be arranged along a circumferential direction of the support cylinder 1410 side by side (uniformly or non-uniformly). The end ring 1430 may be arranged around the plurality of leg antennas 1420 and form an emission coil with the plurality of leg antennas 1420 to emit RF pluses/signals to the target subject 1200.

In some embodiments, one or more acquisition coils 1400 (e.g., the same as or similar to the acquisition coils 1021) may be arranged on the support cylinder 1410 and/or the end ring 1430 for collecting interference signals. In comparison with the coil assembly 300 or 1000, the support cylinder 1410 and the end ring 1430 may be easier to mount the acquisition coils 1400, and the acquisition coils 1400 may be closer to the outside of the MRI device 1401, such that the acquisition coils 1400 can fully receive interference signals in the magnetic field environment.

FIG. 15 is a schematic diagram illustrating an exemplary radial view of the support cylinder 1410 according to some embodiments of the present disclosure. As shown in FIGS. 14 and 15, one or more acquisition coils 1400 may be arranged on the external surface of the support cylinder 1410. When a plurality of acquisition coils 1400 are arranged on the external surface of the support cylinder 1410, the plurality of acquisition coils may be distributed along the circumferential direction of the support cylinder 1410 uniformly. As shown in FIG. 15, eight acquisition coils 1400 (including four acquisition coils 1400 at each of two ends of the support cylinder 1410) may be arranged on the external surface of the support cylinder 1410. The eight acquisition coils may be arranged in two circles on the external surface of the support cylinder 1410. For example, two adjacent acquisition coils 1400 in each circle may be arranged at an interval of a preset angle α (e.g., 90 degrees). According to the uniform arrangement of the acquisition coils 1400, the acquisition range of the acquisition coils 1400 may be improved (e.g., enlarged).

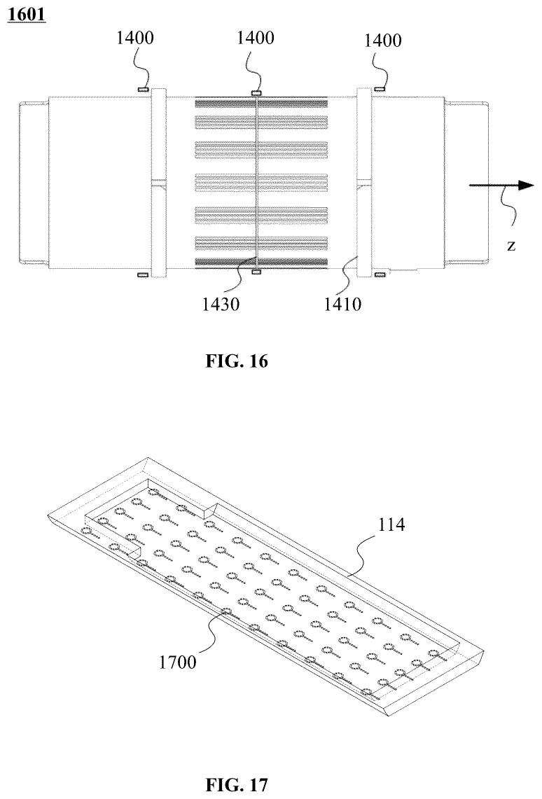

FIG. 16 is a schematic diagram illustrating an exemplary sectional view of a part of an MRI device 1601 along the axial direction (i.e., the z-direction) of the MRI device 1601 according to some embodiments of the present disclosure. Different from the MRI device 1401 as shown in FIG. 14, one or more acquisition coils 1400 of the MRI device 1601 may be arranged on both the external surface of the support cylinder 1410 and the end ring 1430. When there is a plurality of acquisition coils 1400, the acquisition coils 1400 on the support cylinder 1410 may be distributed along the circumferential direction of the support cylinder 1410 uniformly, and the acquisition coils 1400 on the end rings 1430 may be distributed along the circumferential direction the end rings 1430 uniformly. As shown in FIG. 16, four acquisition coils 1400 may be arranged on the external surface of the support cylinder 1410 in two circles. Two acquisition coils 1400 in each circle may be arranged at an interval of 180 degrees. According to the uniform arrangement of the acquisition coils 1400, the acquisition range of the acquisition coils 1400 may be improved (e.g., enlarged).

FIG. 17 is a schematic diagram illustrating an exemplary couch according to some embodiments of the present disclosure. The couch 114 may be provided with one or more acquisition coils 1700 (which may be the same as or similar to the acquisition coils 1400 or 1021). For example, there may be a plurality of acquisition coils 1700 arranged along the axial direction (i.e., the z-direction) of the bore of the MRI device 110, and the plurality of acquisition coils 1700 may be distributed in arrays. When the acquisition coils 1700 are arranged on or within the couch 114, the one or more acquisition coils 1021 arranged on the second housing 1012 of the coil assembly 1000 as shown in FIG. 10B may be omitted, which can reduce the manufacturing difficulty and design cost of the coil assembly 1000.

FIG. 18 is a schematic diagram illustrating an exemplary processing device according to some embodiments of the present disclosure. The processing device 140 may include an obtaining module 1810, a determination module 1820, and a correction module 1830.

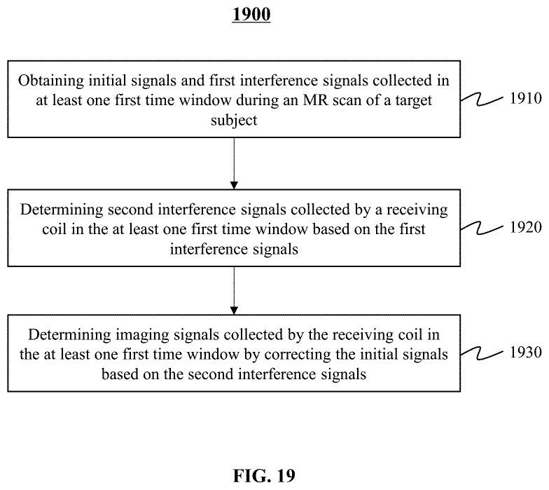

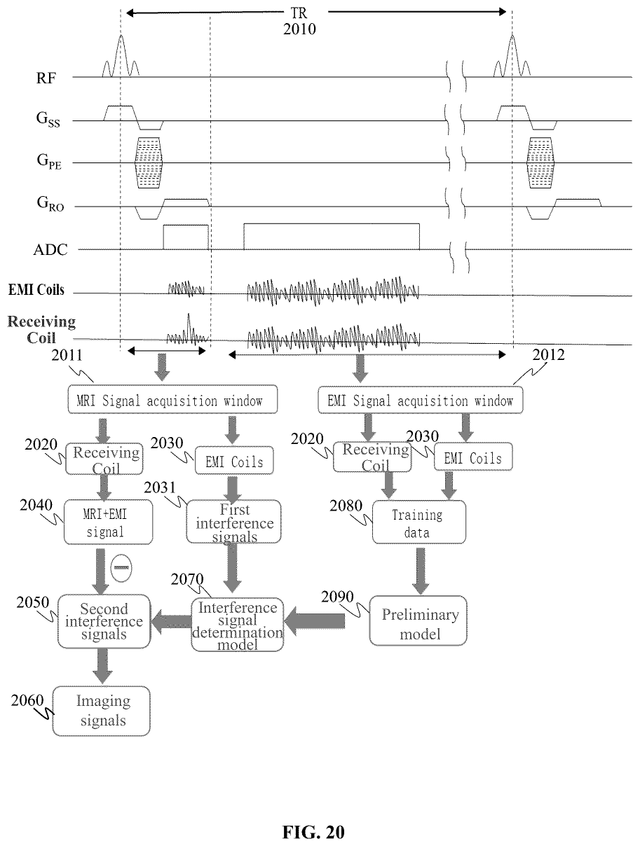

The obtaining module 1810 may be configured to obtain data/information related to signal correction. For example, the obtaining module 1810 may obtain initial signals and first interference signals collected in at least one first time window during an MR scan of a target subject, more descriptions of which can be found elsewhere in the present disclosure (e.g., operation 1910 in FIG. 19 and relevant descriptions thereof). As another example, the obtaining module 1810 may obtain (or generate) an interference signal determination model based on first sample interference signals and second sample interference signals collected in at least one second time window during the MR scan of the target subject, more descriptions of which can be found elsewhere in the present disclosure (e.g., operation 1920 in FIG. 19, FIGS. 20-23 and relevant descriptions thereof).

The determination module 1820 may be configured to determine second interference signals collected by a receiving coil in the at least one first time window based on the first interference signals. For example, the determination module 1820 may determine the second interference signals by using the interference signal determination model. More descriptions regarding the determination of the second interference signals can be found elsewhere in the present disclosure (e.g., operation 1920 in FIG. 19, FIGS. 20, 22, and 23, relevant descriptions thereof).

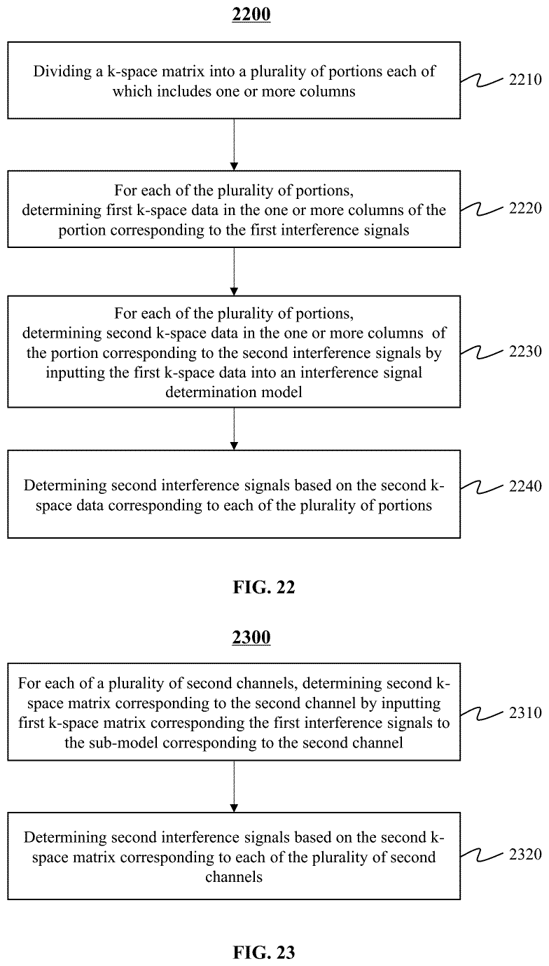

The correction module 1830 may be configured to determine imaging signals collected by the receiving coil in the at least one first time window by correcting the initial signals based on the second interference signals. For example, the correction module 1830 may determine the imaging signals by subtracting the second interference signals from the initial signals. More descriptions regarding the determination of the imaging signals can be found elsewhere in the present disclosure (e.g., operation 1930 in FIG. 19, FIG. 20, FIGS. 22 and 23, and relevant descriptions thereof).

The modules in the processing device 140 may be connected to or communicated with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof. Two or more of the modules may be combined into a single module, and any one of the modules may be divided into two or more units. For example, the above-mentioned modules may be integrated into a console (not shown). Via the console, a user may set parameters for scanning the target subject, controlling imaging processes, controlling parameters for image correction and/or reconstruction, viewing images, etc. As another example, the processing device 140 may include a storage module (not shown) configured to store information and/or data associated with the above-mentioned modules.

FIG. 19 is a flowchart illustrating an exemplary process for signal correction according to some embodiments of the present disclosure.

In 1910, the processing device 140 (e.g., the obtaining module 1810) may obtain initial signals and first interference signals collected in at least one first time window during an MR scan of a target subject.

In some embodiments, the nuclei in the target subject (e.g., the subject 115, the target subject 340, or the target subject 1200) may be in an excited state in the at least one first time window. For example, during the MR scan, the processing device 140 may control the MRI device 110 to apply a scan sequence to excite the nuclei in the target subject. As used herein, a first time window may refer to a time period when the MRI device 110 emits RF signals for exciting the nuclei in the target subject. For example, the first time window may be an MR signal acquisition window 2011 as illustrated in FIG. 20.

In some embodiments, the initial signals may be collected by a receiving coil of the MRI device 110. The receiving coil may include a VTC or a local acquisition coil of the MRI device 110, such as the receiving coil of the coil assembly 300 or the receiving coil of the coil assembly 1000. For example, the receiving coil may include a plurality of second channels. As used herein, a second channel may refer to a coil unit of the receiving coil, more descriptions of which can be found elsewhere in the present disclosure (e.g., FIG. 3 and the relevant descriptions thereof). That is, the receiving coil may include a plurality of coil units. In some embodiments, during the MR scan, after the nuclei of the target subject is excited, the receiving coil may be controlled to collect the initial signals in the least one the first time window. The initial signals may include MR signals (also referred to as imaging signals) received by the receiving coil in the at least one first time window and interference signals (e.g., electromagnetic interference (EMI) signals) received by the receiving coil in the at least one first time window. In some embodiments, after the receiving coil collects the initial signals, the initial signals may be stored in a storage device (e.g., the storage device 150) of the signal collection and processing system 100 of the signal collection and processing system 100. The processing device 140 may retrieve the initial signals from the storage device.

In some embodiments, the first interference signals may be collected by an interference signal acquisition device (e.g., the interference signal acquisition device 210, the interference signal acquisition device of the coil assembly 300 or 1000, etc.) in the at least one first time window. The interference signal acquisition device may include a plurality of first channels. As used herein, a first channel of the interference signal acquisition device may refer to an acquisition unit of the interference signal acquisition device. That is, the interference signal acquisition device may include a plurality of acquisition units. Each acquisition unit of the interference signal acquisition device may include an acquisition coil, more descriptions of which can be found elsewhere in the present disclosure (e.g., FIGS. 2-17 and the relevant descriptions thereof). For example, during the MR scan, the interference signal acquisition device may be controlled to receive the first interference signals (e.g., the EMI signals) in the at least one first time window. In some embodiments, after the interference signal device collects the first interference signals, the first interference signals may be stored in a storage device (e.g., the storage device 150) of the signal collection and processing system 100. The processing device 140 may retrieve the first interference signals from the storage device.

In 1920, the processing device 140 (e.g., the determination module 1820) may determine second interference signals collected by the receiving coil in the at least one first time window based on the first interference signals.

As used herein, the second interference signals collected by the receiving coil in the at least one first time window may be an estimation of the interference signals collected by the receiving coil in the at least one first time window.

In some embodiments, the processing device 140 may determine a relationship (e.g., a mathematical relationship) between the first interference signals and the second interference signals. The processing device 140 may generate the second interference signals based on the first interference signals and the relationship between the first interference signals and the second interference signals. For example, the relationship between the first interference signals and the second interference signals may be obtained/expressed by an interference signal determination model. That is, the processing device 140 may determine the second interference signals based on the first interference signals using the interference signal determination model. For instance, the processing device 140 may input the first interference signals into the interference signal determination model. The processing device 140 may determine the second interference signal based on an output of the interference signal determination model. More descriptions regarding the determination of the second interference signals can be found elsewhere in the present disclosure (e.g., FIGS. 22 and 23 and the descriptions thereof).

In some embodiments, the interference signal determination model may include a trained machine learning model. For example, the trained machine learning model may include a convolutional neural network (CNN) model, a recurrent neural network (RNN) model, a generative adversarial network (GAN) model, a long short-term memory (LSTM) network model, an automatic encoder network model, a deep belief network (DBN) model, a deep residual network model, a gate recurrent unit (RGU) network model, an echo state network model, or the like, or any combination thereof.

In some embodiments, the interference signal determination model may be trained based on first sample interference signals and second sample interference signals collected in at least one second time window during the MR scan. The nuclei in the subject may be in an unexcited state in the at least one second time window. As used herein, a second time window may refer to a time period when the MRI device 110 is not emitting RF signals and the nuclei in the target subject is not excited. In some embodiments, if the transverse magnetization vector of the protons in the target subject is in a preset state (e.g., an initial state), the nuclei in the target subject may be determined to be in an unexcited state.

For example, the second time window may be an EMI signal acquisition window 2012 as illustrated in FIG. 20. The first sample interference signals may be collected by the receiving coil of the MRI device, and the second sample interference signals may be collected by the interference signal acquisition device. In some embodiments, each of at least one second time window may correspond to one of the at least one first time window. The first time window and its corresponding second time window may constitute a repletion time (TR) cycle of the scan sequence (i.e., an interval between two excitation pulses in the scan sequence). Each of the at least one first time window may correspond to an interference signal determination model. The interference signal determination model corresponding to a specific first time window may be trained based on sample interference signals collected in a corresponding second time window of the specific first time window. In some embodiments, the corresponding relationship between the at least one first time window and the at least one second time window may be adjusted according to an actual situation. Merely by way of example, a second time window may correspond to multiple first time windows. Sample interference signals collected in the second time window may be used to generate an interference signal determination model, which may be used for determining interference signals collected in the multiple first time windows.

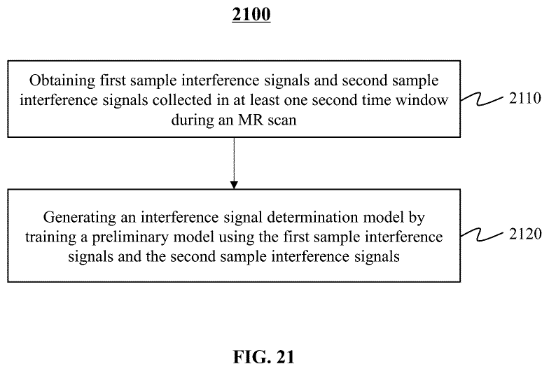

In some embodiments, the interference signal determination model may be trained by a training module of the processing device 140. Alternatively, the trained interference signal determination model may be trained by another processing device different from the processing device 140. More descriptions regarding the training of the trained interference signal determination model can be found elsewhere in the present disclosure (e.g., FIGS. 21-23 and the relevant descriptions thereof).

In 1930, the processing device 140 (e.g., the correction module 1830) may determine imaging signals (also referred to as target imaging signals) collected by the receiving coil in the at least one first time window by correcting the initial signals based on the second interference signals.

As used herein, the imaging signals collected by the receiving coil in the at least first time window may refer to corrected initial signals including fewer interference signals compared with the initial signals, for example, the corrected initial signals may include no interference signals or with little interference signals.

In some embodiments, the processing device 140 may generate the imaging signals by removing the second interference signals from the initial signals, thereby achieving interference signal shielding or correction. For example, the processing device 140 may subtract the second interference signals from the initial signals to generate the imaging signals. Further, the processing device 140 may perform a reconstruction operation on the imaging signals and determine MR image(s) of the target subject.

According to some embodiments of the present disclosure, the target subject may be placed in the main magnetic field environment, and the MRI device 110 may be controlled to perform a scan sequence to excite the nuclei in the target subject. The initial signals and the first interference signals may be collected at the same time period. The second interference signals collected by the receiving coil in the time period may be predicted based on the first interference signals. The second interference signals may be used for correcting the initial signals to determine the target imaging signals. In this way, the interference signals in the initial signals may be predicted and removed, thereby eliminating or reducing the interference of the interference signals on the resulting MR image(s).

It should be noted that the initial signals, the first interference signals, and the second interference signals may be data in the image domain or data in the frequency domain (e.g., k-space data).

It should be noted that the descriptions regarding the signal correction process are for illustration purposes, and not intended to limit the scope of the present disclosure. For persons having ordinary skills in the art, multiple variations and modifications may be made under the teachings of the present disclosure. In some embodiments, the process 1900 may include one or more additional operations or one or more operations in the process 1900 may be omitted. For example, after operation 1930, the process 1900 may include an additional operation for storing the imaging signals for further processing (e.g., image reconstruction).

FIG. 20 is a schematic diagram illustrating an exemplary process of correcting EMI signals according to some embodiments of the present disclosure. As shown in FIG. 20, in an MR scan sequence, RF excitation pulses are used to excite the nuclei in a target subject to generate MR signals. A stratified gradient magnetic field is used to select tissue laminates. Encoding gradient magnetic field is used to perform gradient encoding to realize signal excitation at different positions. A signal readout gradient field is used to cooperate with a data acquisition window for collecting signals.

As shown in FIG. 20, during a repetition time (TR) cycle 2010 of an MR scan sequence (i.e., a time interval between two adjacent RF pluses), there are two time windows (e.g., an MRI signal acquisition window 2011 and an EMI signal acquisition window 2012). During the MR signal acquisition window 2011 (or referred to as a first time window), the RF pulse emission, the gradient emission, and the collection of initial signals are performed. During the EMI signal acquisition window 2012 (or referred to as a second time window), the transverse magnetization vector of the target subject returns to an initial state.

During the MR signal acquisition window 2011, initial signals 2040 collected by a receiving coil 2020 (which is the same as or similar to the receiving coil in the present disclosure) include MRI signals and EMI signals. Therefore, EMI coils 2030 (which are the same as the acquisition coils of the interference signal acquisition device 210, the interference signal acquisition device of the coil assembly 300, the interference signal acquisition device of the coil assembly 1000, etc.) are used to collect first interference signals 2031 for correcting the initial signals 2040. That is, the initial signals 2040 and the first interference signals 2031 are both collected during the MR signal acquisition window 2011. The initial signals 2040 and the first interference signals 2031 may be regarded as being collected synchronously. The first interference signals 2031 are further used to determine second interference signals 2050 by using an interference signal determination model 2070. Further, the second interference signals 2050 is used to correct the initial signals 2040 to determine imaging signals 2060 (or referred to as EMI-free MRI signals).

The training process of the interference signal determination model 2070 may include operations as follows. Sample interference signals collected by the EMI coils 2030 and the receiving coil 2020 within the EMI signal acquisition window 2012 are used as training data 2080 to train a preliminary model 2090 to obtain the interference signal determination model 2070. The sample interference signals collected by the EMI coils 2030 in the EMI signal acquisition window 2012 are used as an input set, and the sample interference signals collected by the receiving coil 2020 in the EMI signal acquisition window 2012 are used as a training label. More descriptions regarding the training process can be found elsewhere in the present disclosure (e.g., FIGS. 21-23 and the descriptions thereof).

FIG. 21 is a schematic diagram illustrating an exemplary process for determining an interference signal determination model according to some embodiments of the present disclosure. In some embodiments, at least a portion of operation 1920 may be achieved by the process 2100. The following descriptions take the implementation of the process 2100 by the determination module 1820 of the processing device 140 for illustration purposes, and the process 2100 may be executed by other module (e.g., a training module) of the processing device 140 or another processing device (e.g., a computing device of a vendor of the interference signal determination model).

In 2110, the processing device 140 (e.g., the determination module 1820) may obtain first sample interference signals and second sample interference signals collected in at least one second time window during an MR scan.

As described in connection with FIGS. 19 and 20, the nuclei in the target subject may be in an unexcited state in the at least one second time window. The first sample interference signals may be collected by the receiving coil of the MRI device 110, and the second sample interference signals may be collected by the interference signal acquisition device.

In 2120, the processing device 140 (e.g., the determination module 1820) may generate an interference signal determination model by training a preliminary model using the first sample interference signals and the second sample interference signals.

In some embodiments, the first sample interference signals may be used as a training input, and the second sample interference signals may be used as a training label. In some embodiments, the processing device 140 may generate the interference signal determination model after the MR scan is finalized. The processing device 140 may generate a single interference signal determination model that can be used to correct the first interference signals collected in different TR cycles.

In some embodiments, each TR cycle of the MR scan may include one first time window and one second time window. For each TR cycle, the processing device 140 may train the preliminary model using the first sample interference signals and the second sample interference signals collected in the second time window of the TR cycle to determine an interference signal determination model corresponding to the TR cycle. The interference signal determination model corresponding to the TR cycle may be used for determining interference signals collected by the receiving coil in the first time window of the TR cycle. That is, an interference signal acquisition model trained using sample interference signals in a second time window may be used to correct initial signals collected in a first time window that belongs to the same TR cycle as the second time window. For example, if there are two TR cycles, i.e., the at least one second time window includes two second time windows, the processing device 140 may determine two interference signal determination models corresponding to the two second time windows respectively. In some embodiments, an interference signal determination model corresponding to a TR cycle may be generated immediately after the TR cycle is finished. In such cases, the interference signals determination model can be trained during the MR scan, such that the initial signals can be corrected while the MR scan is performed, thereby improving the correction efficiency of the initial signals.

In some embodiments, an interference signal determination model determined in a previous TR cycle may be used as a preliminary model of a later TR cycle. For example, if there are two TR cycles, i.e., the at least one second time window includes two second time windows (e.g., a second time window in a first TR cycle and a second time window in a second TR cycle). An interference signal determination model corresponding to the first TR cycle (also referred to as a first trained model) may be generated by training the preliminary model using sample interference signals collected in the second time window in the first TR cycle. An interference signal determination model corresponding to the second TR cycle may be generated by training the first trained model using sample interference signals collected in the second time window in the second TR cycle. In such cases, the training efficiency and the accuracy of the interference signal acquisition model corresponding to the second TR cycle can be improved.

In some embodiments, an interference signal determination model may include a first model and a second model. The first model may be configured to determine amplitude information of the second interference signals by processing amplitude information of the first interference signals. The second model may be configured to determine phase information of the second interference signals by processing phase information of the first interference signals. In such cases, in the training process of the interference signal determination model, the processing device 140 may determine first amplitude information and first phase information of the first sample interference signals. The processing device 140 may determine second amplitude information and second phase information of the second sample interference signals. The processing device 140 may generate the first model by training a first preliminary model using the first amplitude information and the second amplitude information. The processing device 140 may generate the second model by training a second preliminary model using the first phase information and the second phase information. The first preliminary model may be the same as or different from the second preliminary model. During the application of the first model and the second model, the amplitude information of the first interference signals may be input to the first model to determine the amplitude information of the second interference signals, and the phase information of the first interference signals may be input to the second model to determine the phase information of the second interference signals. In such cases, the first model and the second model may be trained individually, thereby reducing or eliminating the interaction between the amplitude information and the phase information in the second interference signals, and improving the accuracy of interference signal determination.

In some embodiments, the processing device 140 may train the interference signal determination model in a cascade manner. For example, the processing device 140 may generate an intermediate model by training a preliminary model using the first amplitude information and the second amplitude information. The processing device 140 may generate the interference signal determination model by training the intermediate model using the first phase information and the second phase information. As another example, the processing device 140 may generate an intermediate model by training a preliminary model using the first phase information and the second phase information. The processing device 140 may generate the interference signal determination model by training the intermediate model using the first amplitude information and the second amplitude information. During the application of the interference signal determination model, the first interference signals may be input into the interference signal determination model to determine the second interference signals. In such cases, the interference signal determination model may be trained in a cascade manner, which can reduce or eliminate the interaction between the amplitude information and the phase information during the training process, thereby improving the accuracy of the interference signal determination model.

In some embodiments, the interference signal acquisition device may include a plurality of first channels (i.e., acquisition units), and the receiving coil may include a plurality of second channels (i.e., coil units). A count of the plurality of second channels may be greater than a count of the plurality of first channels. Merely by way of example, the interference signal acquisition device may include 12 first channels, and the receiving coil may include 24 second channels.

In some embodiments, the first interference signals collected by the interference signal acquisition device in the at least one first time window may include or be transformed into interference k-space data collected by the first channels, wherein the interference k-space data collected by each first channel may include multiple columns (or rows) of k-space data corresponding to a k-space matrix. The second interference signals to be determined may include predicted k-space data collected by the second channels in the at least one first time window, wherein the predicted k-space data collected by each second channel may also include multiple columns of k-space data corresponding to the k-space matrix. Normally, the k-space matrix is large, and directly using the interference signal determination model to process all the interference k-space data corresponding to the first interference signals and/or output all the predicted k-space data corresponding to the second interference signals may cost a lot of computational resources and time. Correspondingly, the training efficiency of the interference signal determination model is low. Therefore, the present disclosure provides methods that process the first interference signals in batches and/or output the second interference signals in batches to improve the processing efficiency. Specifically, the process 2200 as shown in FIG. 22 may be performed.

FIG. 22 is a schematic diagram illustrating an exemplary process for determining second interference signals according to some embodiments of the present disclosure. In some embodiments, at least a portion of the operation 1920 may be achieved by the process 2200.

In 2210, the processing device 140 (e.g., the determination module 1820) may divide a k-space matrix into a plurality of portions each of which includes one or more columns.

The k-space matrix may refer to a matrix corresponding to the k-space to be filled by signals collected by the receiving coil or the interference signal acquisition device. In some embodiments, the size of the k-space matrix may relate to the number of frequency encoding steps and the number of phase encoding steps performed in the MR scan. For example, the size of the k-space matrix may be 128×128 or 256×256.

In some embodiments, the k-space matrix may be evenly divided into the portions, that is, different portions include the same number of columns. For example, the k-space matrix includes 128 columns, and the k-space matrix may be evenly divided into 128 portions each of which includes one column. As another example, the k-space matrix may be evenly divided into 16 portions each of which includes 8 columns.

In 2220, for each of the plurality of portions, the processing device 140 (e.g., the determination module 1820) may determine first k-space data in the one or more columns of the portion corresponding to the first interference signals.

In some embodiments, the first interference signals may include first interference signals collected by each first channel of the interference signal acquisition device. For each first channel, the processing device 140 may fill the first interference signals collected by the first channel into the K-space matrix to determine a filled k-space matrix (i.e., the interference k-space data) corresponding to the first channel. For each of the plurality of portions, the processing device 140 may determine first k-space data in the one or more columns of the portion from the filled k-space matrix of each first channel. For example, it is assumed that there are 12 first channels, and each portion includes one column in the k-space matrix. In such cases, 12 filled k-space matrixes may be generated, and the first k-space data of each portion includes 12 columns each of which is acquired from one of the 12 filled k-space matrixes. For example, for a portion including the first column of the k-space matrix, the first K-space data of the portion may include k-space data in the first column in each of the 12 filled k-space matrixes.

In 2230, for each of the plurality of portions, the processing device 140 (e.g., the determination module 1820) may determine second k-space data in the one or more columns of the portion corresponding to the second interference signals by inputting the first k-space data into an interference signal determination model.

As used herein, the interference signal determination model may be configured to receive the first k-space data in the one or more columns in each of the plurality of portions and output the second K-space data in the one or more columns in each of the plurality of portions. That is, an input of the interference signal determination model may include a portion of the first interference signals, and an output of the interference signal determination model may include a portion of the second interference signals.

In some embodiments, for a specific portion, the second k-space data may include predicted k-space data collected by each second channel in the at least one first time window corresponding to the one or more columns in the specific portion. For example, referring to the example in operation 2220 again, it is further assumed that the receiving coil includes 24 second channels. In such cases, there are 24 filled k-space matrixes corresponding to the 24 second channels to be determined. For the portion including the first column of the k-space matrix, the processing device 140 may input 12 columns of k-space data corresponding to 12 first channels of the interference signal acquisition device into the interference signal determination model, and the interference signal determination model may output k-space data in the first column of each of the 24 filled k-space matrix (i.e., output 24 columns of k-space data each of which corresponds to one second channel).

In 2240, the processing device 140 (e.g., the determination module 1820) may determine second interference signals based on the second K-space data corresponding to each of the plurality of portions.

In some embodiments, the processing device 140 may determine the second interference signals by combining the second k-space data corresponding to each of the plurality of portions. For example, the second k-space data may be rearranged in k-space based on the columns in each portion, and the rearranged k-space data may be regarded as the second interference signals. In some embodiments, for each second channel, the second k-space data of the second channel may be rearranged to generate the corresponding filled k-space matrix, which may be regarded as the second interference signals collected by the second channel in the at least one first time window.

According to some embodiments of the present disclosure, by dividing the k-space matrix into multiple portions, and using the interference signal determination model to process the first interference signals in batches and output the second interference signals in batches, the processing efficiency of the interference signal determination may be improved, thereby improving the whole efficiency of the correction process. It should be understood that in the training process of the interference signal determination model, the training input may also be divided into multiple portions and the training output may include a portion of prediction data.

FIG. 23 is a schematic diagram illustrating an exemplary process for determining second interference signals according to some embodiments of the present disclosure. In some embodiments, at least a portion of the operation 1920 may be achieved by the process 2300.

In some embodiments, as the plurality of first channels may have different impacts on each of the plurality of second channels, the interference signal determination mode may include a plurality of sub-models each of which corresponds to one of the plurality of second channels. A sub-model corresponding to a second channel may be configured to receive first interference signals collected by the plurality of first channels and output second interference signals corresponding to the second channel.

In 2310, for each of the plurality of second channels, the processing device 140 (e.g., the determination module 1820) may determine a second k-space matrix corresponding to the second channel by inputting a first k-space matrix corresponding to the first interference signals to the sub-model corresponding to the second channel.

In some embodiments, for each of the plurality of first channels, the processing device 140 may determine a first k-space sub-matrix by filling the first interference signals collected by the first channel into k-space. The first k-space matrix may include a plurality of first k-space sub-matrixes corresponding to the plurality of first channels. The processing device 140 may input the plurality of first k-space sub-matrixes together into the sub-model corresponding to the second channel to determine the second k-space matrix corresponding to the second channel. In such cases, the sub-model corresponding to the second channel may be trained using first sample k-space matrixes corresponding to first sample interference signals collected by the plurality of first channels as a training input and second sample k-space matrixes corresponding to second sample interference signals collected by the second channel as a training label.

In some embodiments, for each point in a second k-space matrix corresponding to a second channel, the processing device 140 may determine data corresponding to the point collected by the plurality of first channels. The processing device 140 may input the data corresponding to each point collected by the plurality of first channels into the sub-model corresponding to the second channel to determine data corresponding to each point in second K-space matrix corresponding to the second channel. That is, an input of the sub-model corresponding to the second channel may include data corresponding to each point in the first k-space sub-matrixes collected by the plurality of first channels.

In 2320, the processing device 140 (e.g., the determination module 1820) may determine second interference signals based on the second k-space data corresponding to each of the plurality of second channels.

In some embodiments, for each of the plurality of second channels, the processing device 140 may determine second interference signals corresponding to the second channel based on the second k-space data corresponding to second channel. For example, for each second channel, the second k-space matrix of the second channel may be regarded as the second interference signals collected by the second channel in the at least one first time window. In some embodiments, each point in the second k-space data may be rearranged in k-space, and the rearranged k-space data may be regarded as the second interference signals.

According to some embodiments of the present disclosure, each of the plurality of second channels may have a corresponding sub-model by considering the interference of each of the plurality of first channels on the second channel, which uses a large amount of data in the training process, such that the accuracy of the sub-model corresponding to the second channel may be improved.

In some embodiments, the processes 1900-2300 may be executed by the signal collection and processing system 100. For example, the processes 1900-2300 may be implemented as a set of instructions (e.g., an application) stored in a storage device (e.g., the storage device 150). The modules described in FIG. 18 may execute the set of instructions and may accordingly be directed to perform the processes 1900-2300. The operations of the illustrated processes presented below are intended to be illustrative. In some embodiments, each of the processes 1900-2300 may be accomplished with one or more additional operations not described and/or without one or more of the operations discussed. Additionally, the order of the operations of each of the processes 1900-2300 is illustrated in FIGS. 19-23 and described below are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method, implemented by a computing device including a storage device and at least one processor, the method including:
obtaining initial signals and first interference signals collected in at least one first time window during a Magnetic Resonance (MR) scan of a target subject, the nuclei in the target subject being in an excited state in the at least one first time window, the initial signals being collected by a receiving coil of a Magnetic Resonance Imaging (MRI) device, and the first interference signals being collected by an interference signal acquisition device;
determining second interference signals collected by the receiving coil in the at least one first time window based on the first interference signals; and
determining imaging signals collected by the receiving coil in the at least one first time window by correcting the initial signals based on the second interference signals.

2. The method of claim 1, wherein the second interference signals are determined based on the first interference signals using an interference signal determination model, the interference signal determination model including a trained machine learning model.

3. The method of claim 2, wherein the interference signal determination model is determined by:
obtaining first sample interference signals and second sample interference signals collected in at least one second time window during the MR scan, the nuclei in the target subject being in an unexcited state in the at least one second time window, the first sample interference signals being collected by the receiving coil of the MRI device, and the second sample interference signals being collected by the interference signal acquisition device; and
generating the interference signal determination model by training a preliminary model using the first sample interference signals and the second sample interference signals.

4. The method of claim 2, wherein
the interference signal determination model includes a first model and a second model,
the first model is configured to determine amplitude information of the second interference signals by processing amplitude information of the first interference signals, and
the second model is configured to determine phase information of the second interference signals by processing phase information of the first interference signals.

5. The method of claim 2, wherein the interference signal acquisition device includes a plurality of acquisition units, and the receiving coil includes a plurality of coil units.

6. The method of claim 5, wherein the determining second interference signals collected by the receiving coil in the at least one first time window based on the first interference signals includes:
dividing a k-space matrix into a plurality of portions each of which includes one or more columns;
for each of the plurality of portions,
determining first K-space data in the one or more columns of the portion corresponding to the first interference signals; and
determining second K-space data in the one or more columns of the portion corresponding to the second interference signals by inputting the first K-space data into the interference signal determination model; and
determining the second interference signals based on the second K-space data corresponding to each of the plurality of portions.

7. The method of claim 5, wherein the interference signal determination model includes a plurality of sub-models each of which corresponds to one of the plurality of coil units, and the determining second interference signals collected by the receiving coil in the at least one first time window based on the first interference signals includes:
for each of the plurality of coil units, determining a second k-space matrix corresponding to the second channel by inputting a first k-space matrix corresponding to the first interference signals to the sub-model corresponding to the second channel; and
determining the second interference signals based on the second K-space matrix corresponding to each of the plurality of coil units.

8. The method of claim 1, wherein
the target subject is accommodated in an accommodation space formed by a housing during the MR scan,
the housing includes a first external surface facing the target subject and a second external surface opposite to the first external surface,
the receiving coil is arranged within the housing between the first external surface and the second external surface of the housing, and
the interference signal acquisition device is arranged outside the housing on the second external surface of the housing or inside the housing.

9. The method of claim 1, wherein
the target subject is accommodated in a detection region of the MRI device during the MR scan,
the receiving coil is arranged within the detection region, and
acquisition units of the interference signal acquisition device are arranged at two ends of the detection region.

10. A coil assembly for Magnetic Resonance Imaging (MRI), comprising:
a housing forming an accommodation space configured to accommodate a target subject;
a receiving coil configured to collect initial signals during an MR scan of the target subject; and
an interference signal acquisition device configured to collect interference signals during the MR scan, wherein the interference signals are used to correct the initial signals to determine imaging signals collected during the MR scan.

11. The coil assembly of claim 10, wherein
the housing includes a first external surface facing the target subject and a second external surface opposite to the first external surface,
the receiving coil is arranged within the housing between the first external surface and the second external surface of the housing, and
the interference signal acquisition device is arranged outside the housing on the second external surface of the housing.

12. The coil assembly of claim 11, wherein the interference signal acquisition device includes one or more acquisition units each of which includes an acquisition coil, a surface of the acquisition coil and a surface of each coil unit of the receiving coil forming a preset angle, and a size of the acquisition coil being smaller than a size of each coil unit of the receiving coil.

13. The coil assembly of claim 12, wherein an axial direction of the acquisition coil is determined based on a direction of a main magnetic field that is used to scan the target subject.

14. The coil assembly of claim 12, wherein the acquisition coil includes a toroidal inductor configured to collect interference signals along a central axis of the toroidal inductor.

15. The coil assembly of claim 12, wherein the interference signal acquisition device further includes a detuned circuit electrically connected to the acquisition coil, the detuned circuit being configured to adjust the acquisition coil to a detuned state or a resonant state.

16. The coil assembly of claim 15, wherein the interference signal acquisition device further includes a preamplifier circuit and a wave trap unit, the preamplifier circuit being electrically connected to the detuned circuit and the wave trap unit.

17. The coil assembly of claim 15, wherein
the second external surface of the housing is provided with one or more slots;
each of the one or more acquisition units is provided with a buckle at a bottom of the acquisition unit, wherein the buckle corresponds to one of the one or more slots and is provided with a through hole through which a connection wire of the interference signal acquisition device is passed to connect the corresponding acquisition coil with the detuned circuit.

18. The coil assembly of claim 12, wherein the interference signal acquisition device further includes one or more mounting bases configured to support the one or more acquisition coils on the second external surface of the housing.

19. The coil assembly of claim 18, wherein
each of the one or more mounting bases includes a support and a mounting part,
the support is mechanically connected to the second external surface of the housing, and
the mounting part is mechanically connected to a portion of the support away from the second external surface and configured to limit a corresponding interference signal acquisition unit to maintain a preset distance from the second external surface of the housing.

20. The coil assembly of claim 10, wherein the housing includes a flexible housing that is deformable to adapt to the surface of the target subject.
